(19) United States
(12) Patent Application Publication
(10) Pub. No.: US 2012/0184008 A1
(43) Pub. Date: Jul. 19, 2012

(54) Filamentous fungi and methods for producing trichodiene from lignocellulosic feedstocks

(75) Inventor: Thomas Hohn, Chapel Hill, NC (US)

(73) Assignee: NOVOZYMES A/S, Bagsvaerd (DK)

(21) Appl. No.: 13/386,894

(22) PCT Filed: Aug. 5, 2010
(86) PCT No.: PCT/US10/44595

§ 371 Date: Apr. 2, 2012

Related U.S. Application Data
(60) Provisional application No. 61/231,374, filed on Aug. 5, 2009.

(51) Int. Cl. C12P 5/00

(52) U.S. Cl. 435/166

(57) ABSTRACT

The present invention relates to the production of a C-15 fuel from lignocellulosic or other feedstock. Specifically at least double mutant of filamentous fungi having the isoprenoid pathway results in production of trichodiene in commercial quantities. One embodiment of the invention relates to producing the fuel at the site of the lignocellulosic feedstock to reduce costs of shipping the feedstock.

The application claims priority of U.S. provisional application No. 61/231,374 filed on Aug. 5, 2009 and is included herein in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to the production of trichodiene from a filamentous fungus using a biomass feedstock such as a lignocellulosic feedstock. In particular, the present invention relates to a filamentous fungi having the trichothecenes pathway and method for producing trichodiene using biomass feedstock wherein the fungus is a mutant fungus having no or low Tri4 expression or Tri4 suppression and increased expression of at least one of Tri5, Tri6 or Tri10.

2. Description of Related Art

Current world dependence on the use of petroleum fuels for transportation presents threats to both the global environment in the form of increased CO₂ levels and the decreased energy security of many countries. The production of liquid transportation fuels from plant materials provides a renewable alternative to petroleum based fuels.

With the development of the biofuels industry over the last several years it has become apparent that two of the key elements for sustainability and economic feasibility are the choice of feedstocks and the properties of the fuel that is produced. For example, in the United States the use of corn as a feedstock for fuel ethanol production has been perceived to have had both direct and indirect negative effects on food and feed commodity prices because of the competing interests. These effects have accelerated efforts to develop non-food related feedstocks such as lignocellulose for biofuel production. The primary research and development into Biofuels has been for the production of ethanol, although important technical and economic barriers to the use of lignocellulosic feedstocks for ethanol production remain unaddressed.

It has also been recognized that chemical properties of the selected biofuel can have a significant impact on biofuel economics. As such, there remains a commercial need for fuel alternatives to ethanol that require lower energy inputs for processing, are better suited for pipeline transport, and have better compatibility with petroleum transportation fuels.

Trichodiene is a cyclic hydrocarbon that was originally isolated from the fungus Trichothecium roseum [S. Nozoe and Y. Machida, Tetrahedron 28: 5105-5111 (1972)]. Current technologies for large scale production of trichodiene and other terpenoids by fungi frequently involve the introduction of heterologous biosynthetic pathways or the manipulation of native pathways via mutagenesis using targeted or non-targeted mechanisms of genetic alteration. However, it is has not been previously economically feasible to produce trichodiene in large quantities.

Fungi capable of accumulating large quantities of sesquiterpene hydrocarbons, such as trichodiene, represent attractive systems for the commercial production of biofuels and other valuable isoprenoid products such as carotenoids. The production of large quantities of sesquiterpenoid mycotoxins such as trichothecenes has been observed for several Fusarium species including F. sporotrichioides, F. graminearum, and F. sambucinum. One of the most prolific of these is F. sporotrichioides which has been reported to produce up to 2.9 g trichothecenes/liter of culture medium (Fusarium sporotrichioides. Curr. Genet. 24:291-295). The introduction of chemical or genetic blocks in the trichothecene pathway designed to inhibit or cause loss-of-function for the second enzymatic step in the pathway (Tri4 gene product) results in the accumulation of the sesquiterpene hydrocarbon, trichodiene. Several chemical inhibitors of Tri4 gene product, including the plant growth regulator compound Ancymidol, are known to result in trichodiene accumulation, while a mutant strain of F. sporotrichioides derived from NRRL 3299 (NRRL 18340) (MB5493) (T-0927) produces only trichodiene and no other trichothecene pathway intermediates. Disruption of the Tri4 gene in F. sporotrichioides by molecular genetic approaches leading to loss of Tri4 function also results in the accumulation of trichodiene.

### BRIEF SUMMARY OF THE INVENTION

The present invention relates to the discovery that a filamentous fungus having the trichothecene biosynthesis pathway which has a lower, non-functioning or inhibited (chemically or biologically) Tri4 gene alone with one or more augmented gene products from the group of Tri5, Tri6 and Tri10 produces an improvement in the production of trichodiene and improves the efficiency for biomass feedstock utilization by producing enzymes, reducing costs, and providing opportunities for small scale production.

Accordingly, in one embodiment of the present invention there is a mutant trichodiene producing filamentous fungus having the trichothecene pathway comprising:
a) a disrupted Tri4 gene or a mutant Tri4 gene having low P450 monooxygenase production; and
b) a modified nucleic acid sequence encoding for at least one of the genes selected from the group consisting of Tri5, Tri6 and Tri10, the sequence modified such that the filamentous fungus produces at least 10% more trichodiene than the parent filamentous fungal cell when cultured under the same conditions.

In another embodiment of the present invention there is disclosed a mutant trichodiene producing filamentous fungus having the trichothecene pathway comprising:
a) a modified nucleic acid sequence encoding for at least one of the genes selected from the group consisting of Tri5, Tri6 and Tri10, the sequence modified to increase the production of the gene product; and
b) the presence of a Tri4 inhibitor sufficient to inhibit at least a portion of the Tri4 gene product;
wherein the filamentous fungus produces at least 10% more trichodiene than the parent filamentous fungal cell when cultured under the same conditions.

In yet another embodiment of the invention there is disclosed a method of producing trichodiene comprising:
a) selecting a mutant filamentous fungus having the trichothecene pathway comprising:
   i. one or more of a disrupted Tri4 gene, a mutant Tri4 gene having low P450 monooxygenase production and the fungus in combination with a Tri4 gene product inhibitor;
   ii. a modified nucleic acid sequence encoding for at least one of the genes selected from the group consisting of Tri5, Tri6 and Tri10, the sequence modified to increase the production of the gene product;
b) cultivating the mutant filamentous fungus using a growth media selected from the group comprising a sugar, a starch, a cellulose and a hemicelluloses; and
c) isolating trichodiene from the growth media;
wherein the filamentous fungus produces at least 10% more trichodiene than the parent filamentous fungus when cultured under the same conditions and using the same growth media.

### BRIEF DESCRIPTION OF THE DRAWINGS

FIG. 1 is a schematic representation of the mevalonate (“MEV”) pathway for the production of isopentenyl pyrophosphate (“IPP”).

FIG. 2 is a schematic representation of the conversion of isopentenyl pyrophosphate (“IPP”) to farnesyl pyrophosphate (“FPP”) and trichodiene in a Tri4⁻ mutant.

FIG. 3 shows a map of expression plasmid genes.

FIG. 4 shows a map of expression plasmid pDOR311.

FIG. 5 shows a map of expression plasmid pDOR312.

FIG. 6 shows a map of expression plasmid pDOR313.

FIG. 6 shows a map of expression plasmid pDOR313.

FIG. 8 shows a map of expression plasmid pDOR315.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

### DEFINITIONS

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

The term “operably linked” refers to a juxtaposition of biological components on a single DNA molecule that are in a relationship permitting them to function in their intended linked manner. For instance, a promoter is operably linked to a nucleotide sequence if the promoter affects the transcription or expression of the nucleotide sequence.

The term “mutant” refers to cells related to a parent cell by a modification of one or more genes involved in the production of trichothecenes, e.g. disruption or deletion of the Tri4 gene such that the Tri4 gene no longer functions. Examples of a physical or chemical mutagenizing agent suitable for the present purpose include ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, and nucleotide analogues. When such agents are used, the mutagenesis is typically performed by incubating the parent cell to be mutagenized in the presence of the mutagenizing agent of choice under suitable conditions, and selecting for mutant cells exhibiting reduced or no expression of the gene.

Modification or inactivation of the gene may be also accomplished by introduction, substitution, or removal of one or more nucleotides in the gene or a regulatory element required for the transcription or translation thereof. For example, nucleotides may be inserted or removed so as to result in the introduction of a stop codon, the removal of the start codon, or a change of the open reading frame. Such a modification or inactivation may be accomplished by site-directed mutagenesis or PCR generated mutagenesis in accordance with methods known in the art. Although, in principle, the modification may be performed in vivo, i.e., directly on the cell expressing the gene to be modified, it is preferred that the modification be performed in vitro as exemplified below.

Alternatively, modification or inactivation of the gene may be performed by established anti-sense techniques using a nucleotide sequence complementary to the nucleic acid sequence of the gene. More specifically, expression of the gene by a filamentous fungal cell may be reduced or eliminated by introducing a nucleotide sequence complementary to the nucleic acid sequence of the gene which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated.

The term “Filamentous fungi” includes all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative. In the methods of the present invention, the filamentous fungal cell may be a wild-type cell or a mutant thereof. Furthermore, the filamentous fungal cell may be a cell which does not produce any detectable trichothecene(s), but contains the genes encoding a trichothecene(s). Preferably, the filamentous fungal cell is an Acremonium, Aspergillus, Aureobasidium, Cryptococcus, Filibasidium, Fusarium (e.g. F. gramineareum, F. sporotrichioides, F. venenatam) Gibberella, Humicola, Magnaporthe, Mucor, Myceliophthora, Myrothecium, Neocallimastix, Neurospora, Paecilomyces, Penicillium, Piromyces, Stachybotrys, Schizophyllum, Talaromyces, Thermoascus, Thielavia, Tolypocladium, Trichoderma, or Trichothecium cell.

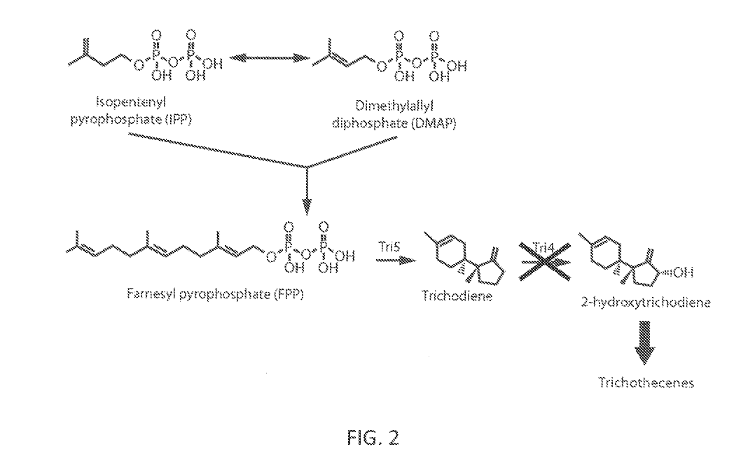

The term “trichothecenes” is defined herein as a family of sesquiterpene epoxides produced by a sequence of oxygenations, isomerizations, cyclizations, and esterifications leading from trichodiene to the more complex trichothecenes. The trichothecenes include, but are not limited to, 2-hydroxytrichodiene, 12,13-epoxy-9,10-trichoene-2-ol, isotrichodiol, isotrichotriol, trichotriol, isotrichodermol, isotrichodermin, 15-decalonectrin, 3,15-didecalonectrin, deoxynivalenol, 3-acetyldeoxynivalenol, calonectrin, 3,15-diacetoxyscirpenol, 3,4,15-triacetoxyscirpenol, 4,15-diacetoxyscirpenol, 3-acetylneosolaniol, acetyl T-2 toxin, and T-2 toxin; and derivatives thereof. The trichothecene biosynthetic pathway is shown in FIG. 2 (Microbiol. Rev., 57: 595-604).

The term “constitutively active” refers to a promoter that is expressed and not known to be subject to regulation completely ceasing expression; that is, it is always “on,” and does not entirely rely on activation by some other biological system.

The term “Inducible” or “Inducibly active” refers to a promoter whose activity level increases in response to treatment with an external signal or agent.

The term “nonrevertable site-selected deletion” refers to the deletion a significant amount of the Tri4 DNA sequences such that the organism is incapable of reversion to the wild type. Reversion is a finite probability over time that exists with naturally occurring or induced point mutations wherein the single mutations could easily naturally mutate back during production use to produce active gene product. Deletions of the invention include large deletions or active site deletions involving a single codon for an active site residue.

The term “gene product” refers to RNA encoded by DNA (or vice versa) or protein that is encoded by an RNA or DNA, where a gene will typically comprise one or more nucleotide sequences that encode a protein, and may also include introns and other non-coding nucleotide sequences.

The term “at least 10% more trichodiene” refers to an increase in the quantity of trichodiene produced by a fungal cell as measured by chemical analytical methods and expressed as grams trichodiene per liter of culture or grams trichodiene per gram fungal culture dry weight when comparing the modified strain to a parent or wild type strain.

The term “enzymatic or catalytic activity” refers to the ability of the Tri4 gene product to catalyze the required chemical transformation of trichodiene so as to produce an oxygenated trichodiene product.

The term “low P450 monooxygenase production” refers to the amount of enzymatically active Tri4 gene product produced in a Tri4 mutant strain or Tri4 inhibited strain such that the levels of trichodiene produced are more than 10% greater than are observed in the parent or wild type strain by chemical analysis under the same growth conditions.

The term “autonomous maintenance” refers to a DNA or vector that replicates within a filamentous fungal cell independently of the chromosomal DNA. For autonomous replication, the DNA or vector may further comprise an origin of replication enabling the vector to replicate autonomously in the filamentous fungal cell in question.

The term “promoter” refers to a portion of a gene containing DNA sequences that provide for binding of RNA polymerase and initiation of transcription and thus refers to a DNA sequence capable of controlling expression of a coding sequence or functional RNA. Promoter sequences are commonly, but not always, found in the 5′ non-coding regions of genes, upstream of one or more open reading frames encoding polypeptides. Sequence elements within promoters that function in the initiation of transcription are often characterized by consensus nucleotide sequences. A promoter sequence may include both proximal and more distal upstream elements. A promoter may be, for example, constitutive, inducible, or environmentally responsive.

The term “terminator” refers to a sequence recognized by a filamentous fungal cell to terminate transcription. The Tri5 terminator sequence is operably linked to the 3′ terminus of the nucleic acid sequences encoding the Tri6 or Tri10 polypeptides. Any terminator which is functional in the filamentous fungal cell may be used in the present invention.

The term “inhibitor” refers to, for purposes of this invention, a substance that prevents an enzymic process as a result of the interaction of the substance with the enzyme so as to decrease the rate of reaction.

The term “trichothecene pathway” is used herein to refer to the biosynthetic pathway that converts farnesyl pyrophosphate (FPP) to trichothecenes. The first two steps in the trichothecene pathway are illustrated schematically in FIG. 2.

The term “glucose equivalent” is used to describe the degree of hydrolysis of starch or cellulose into glucose monomers or the percentage of the total solids that have been or can potentially be converted to reducing sugars.

The term “biomass” refers to any biological material that can used for biofuel or bioproduct industrial processes including but not limited to lignocellulose, algae, algal process wastes, chitin, chitosan, pectins (including sugar beet process residues), and proteins (including oil seed crushing residues). Other materials are known in the art and can be identified by one skilled in the art.

The term “lignocellulosic feedstock” refers to use of plant biomass composed of lignocellulose (cellulose, hemicellulose, and lignin) as a feedstock for biofuel and bioproduct industrial processes. The carbohydrate polymers of lignocellulose (cellulose and hemicelluloses) are tightly bound to the lignin and are not readily accessible to enzymatic hydroloysis. Lignocellulosic feedstocks include but are not limited to agricultural residues (including corn stover, wheat straw, and sugarcane bagasse), energy crops (including sorghum, switchgrass and miscanthus), wood residues (including sawmill and paper mill discards), forestry wastes, industrial wastes (including paper sludge), and municipal paper and landscape waste. Other materials are known and can be identified by one skilled in the art.

The term “vector” refers to a nucleic acid sequence or molecule (e.g. a plasmid) that transduces, transforms, or infects a host strain, thereby causing the cell to produce nucleic acids and/or proteins other than those that are native to the cell, or to express nucleic acids and/or proteins in a manner that is not native to the cell. Alternatively, the vector may contain additional nucleic acid sequences for directing integration by homologous recombination into the genome of the filamentous fungal cell. The additional nucleic acid sequences enable the vector to be integrated into the genome at a precise location(s) in the chromosome(s). To increase the likelihood of integration at a precise location, the integrational elements should preferably contain a sufficient number of nucleic acids, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequences that are homologous with the target sequence in the genome of the filamentous fungal cell. Furthermore, the integrational elements may be non-encoding or encoding nucleic acid sequences. On the other hand, the vector may be integrated into the genome of the cell by non-homologous recombination.

The term “growth media culture” refers to cultivation in a nutrient medium suitable for production of trichodiene using methods known in the art. For example, the cell may be cultivated by shake flask cultivation, or small-scale or large-scale fermentation (including continuous, batch, fed-batch, or solid state fermentations) in laboratory or industrial fermentors with a suitable medium and under conditions allowing the trichodiene to be secreted and/or isolated. Suitable nutrient media comprising carbon and nitrogen sources and inorganic salts are available from commercial suppliers or may be prepared using biomass as the medium carbon source. Those skilled in the art can produce appropriate cultures with minimal experiments in view of the present invention.

The term “parent strain” refers to a strain of microorganism that is mutated, electroporated, or otherwise changed to provide a strain or host strain of the invention, or a strain that precedes a strain that has been mutated, electroporated, or otherwise changed to provide a strain or host strain of the invention.

The term “modified nucleic acid sequence” refers to a nucleic acid molecule, either single- or double-stranded, which is isolated from a naturally occurring gene or which has been modified to contain segments of nucleic acid which are deleted, combined and/or juxtaposed in a manner which would not otherwise exist in nature.

The word “pyrophosphate” is used interchangeably herein with “diphosphate”.

The term “host strain” is used herein to refer to any archae, bacterial, or eukaryotic living cell into which a heterologous nucleic acid can be or has been inserted. The term also relates to the progeny of the original cell, which may not necessarily be completely identical in morphology or in genomic or total DNA complement to the original parent, due to natural, accidental, or deliberate mutation.

The term “transformation” refers to a permanent or transient genetic change induced in a cell following introduction of new nucleic acid. Genetic change (“modification”) can be accomplished either by incorporation of the new DNA into the genome of the host strain, or by transient or stable maintenance of the new DNA as an episomal element. In eukaryotic cells, a permanent genetic change is generally achieved by introduction of the DNA into the genome of the cell.

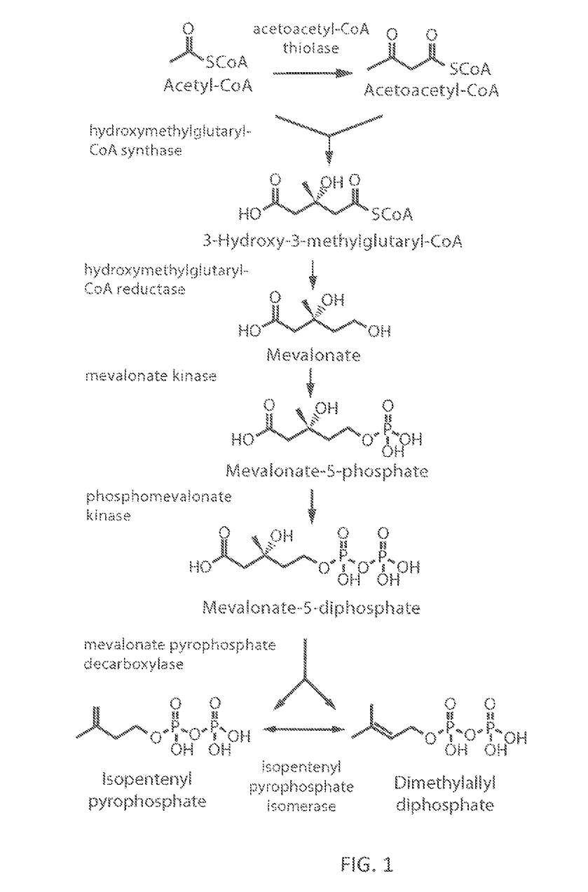

The Trichothecene biosynthetic pathway in filamentous fungi is fairly well known to those skilled in the art. The depictions in FIG. 1 and FIG. 2 outline the trichodiene synthetic pathway as well as its place in the isoprenoid synthetic pathway. FIG. 2 also depicts the known fuel product production using the pathway that is the focus of the present invention. This pathway exists in a number of filamentous fungi including but not limited to species such as Acremonium, Aspergillus, Aureobasidium, Cryptococcus, Filibasidium, Fusarium, Gibberella, Humicola, Magnaporthe, Mucor, Myceliophthora, Myrothecium, Neocallimastix, Neurospora, Paecilomyces, Penicillium, Piromyces, Stachybotrys, Schizophyllum, Talaromyces, Thermoascus, Thielavia, Tolypocladium, Trichoderma, or Trichothecium,

In one embodiment the Filamentous fungus is F. sporotrichioides such as NRRL 3299. In this pathway the production of farnesyl pyrophosphate (FPP) is conserved in these fungi and the Tri5 gene product, trichodiene synthase, is responsible in this pathway for converting FPP to Trichodiene. Trichodiene is a C-15 (15 carbon atoms) bi-cyclic hydrocarbon which if produced in sufficient quantities greater than 0.1 g, 0.22 g or 0.25 g per gram of glucose or glucose equivalent consumed could be considered for utilization as a commercial source of C-15 hydrocarbon fuel. In one embodiment the C-15 fuel is a diesel and/or jet fuel. The production of the Tri5 gene product is regulated at least in part by the Tri6 gene product which is a positive transcription factor controlling the expression of the Tri5 gene product and FPP synthase in the isoprenoid pathway. The Tri10 gene produces a product which is a positive regulator for Tri5, Tri6 and FPP synthase in the Isoprenoid pathway. Both Tri6 and Tri10 appear to control the expression of FPP Synthase, HMG CoA reductase synthase, and Mevalonate kinase and the rest of the pathway enzymes of the isoprenoid pathway and are responsible for upregulating the flow of intermediates into the trichothecene pathway. In addition both Tri6 and Tri10 are known to be active in the regulation of Tri4 and Tri 5. Introducing multiple copies of these genes in a native strain background gives high levels of production for “trichothecenes”, while the interruption or enhancement of the Tri6, Tri5 and Tri10 genes have prior to the present invention not been shown let alone shown in combination with a Tri4 mutant. Prior to the present invention there has been no indication that combinations of these modifications would either work together let alone produce an improved or synergistic effect on the production of trichodiene.

Tri4 gene encodes for the production an enzyme for the conversion of trichodiene to 2-hydroxytrichodiene in the trichothecene biosynthetic pathway. The enzyme P450 monooxygenase becomes the rate limiting step in the conversion of trichodiene. The Tri4 gene is also regulated by Tri6 and Tri10. The isolation and characterization of Tri4, Tri5, Tri6 and Tri10 has shown that they all reside on a 10 kb DNA fragment in a gene cluster in F. sporotrichioides. It is known that they are located in similar positions in other trichothecene producing filamentous fungi.

The present invention relates to the production of C-15 hydrocarbons (that can be used for jet fuel and diesel fuel production) in a filamentous fungus having the isoprenoid pathway in sufficient quantities to be of commercial significance. By combining the disruption (biological or chemical) or partial blockage of Tri4 with at least one other modification in Tri5, Tri6 or Tri10 which either leads to increased trichodiene production or reduced trichodiene conversion to 2-hydroxytrichodiene (by reducing the regulation of Tri4 by Tri6 or Tri10) commercial quantities of trichodiene can be produced and isolated for the Isoprenoid pathway in a filamentous fungus. The modification can be the addition or deletion of all or a portion of the genes, the substitution of other genes, for example, genes found to have constitutive activity or any other modification known in the art to increase the production or activity or other property of the gene as necessary. The production in this species would then represent a tremendous improvement over production bacteria or other species since it can occur under aerobic conditions and the fuel product undergoes a phase separation with water making the process more cost efficient to deploy on small scale production facilities such as an on-farm trichodiene production facilities or other location where the sugar or lignocellulosic material (a biomass) resides. In addition, since most of these Fungal species are able to utilize a number of different biomass feedstock such as cellulose, hemicelluloses sugar sources, algae protein, algae polysaccharides, and the like for production, they represent a practical improvement which allows use of lignocellulosic feedstocks without the substantial addition of processing enzymes for the conversion to component sugars or lignocellulosic stock which usually make other processes too costly and labor intensive. A filamentous fungal production system greatly reduces the need for enzymes, if not eliminates it, thus providing a novel practical solution to biological production of fuels because it could be produced on a small scale locally and it could easily provide an effective solution to the problem of feedstock transportation costs and logistics which can be a bigger barrier in some cases than the production of the fuel itself for any method.

The present invention filamentous fungi have the Tri4 gene modified to reduce or eliminate the production of the Tri4 gene product P450 monooxygenase. Without this enzyme trichodiene is not converted in the next step of the conversion process. It is clear that a chemical modification that blocks the utility of the enzyme or its production would serve the same purpose and is considered part of the means for blocking the production or activity of the enzyme.

The Tri4 modification/treatment is then combined with at least one modification to the Tri5, Tri6 or Tri10 gene/gene product such that even larger quantities of trichodiene can be produced. It has been determined that at least a dual mutant produces more trichodiene than any of the single mutants and in some cases synergistically so. It is difficult to produce these mutants and absent applicant's disclosure it would not have been known that one could achieve such mutants or that they would work to improve trichodiene production to a commercial level. Obviously multiple mutations in the genes could be combined as well to give even higher production of trichodiene.

The modifications to the Tri4 gene are known. The modifications to the other gene sequences can be achieved by any of the known methods for gene modification to increase or decrease the activity of a gene product or the like. One skilled in the art armed with the knowledge of producing the dual mutants could easily without undue experimentation make such dual mutants.

Now referring to the drawings. FIG. 2 is a flow chart choosing the trichodiene production route in filamentous fungi having the isoprenoid production pathway. As can be seen Farnesyl pyrophosphate is reacted on by the Tri5 gene product to produce trichodiene. The Tri4 gene product then reacts with trichodiene to produce 2-hydroxytrichodiene which is further metabolized to trichothecenes. The Tri6 and Tri10 gene products act as regulatory controls in this pathway and hence their combination with modifications to the production of the Tri4 product leads to commercial quantities of trichodiene being produced.

In FIG. 1 there is a general flow chart of the isoprenoid biosynthetic pathway. While gasoline, diesel, and jet fuel type products are produced in this pathway the present invention relates primarily to production of diesel and jet fuels.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1

The filamentous fungus Fusarium sporotrichioides NRRL 3299 is selected with a deleted sequence for Tri4 and thus, cannot produce the Tri4 gene product. The accumulation of trichodiene is observed. This organism is treated to modify the Tri6 gene to have constitutive activity, thus increasing the production of FPP and further increasing trichodiene production.

### Example 2

The NRRL 3299 is again modified, this time both the Tri6 and Tri10 gene are modified such that the Tri5 gene product is increased in production. In a related example the Tri6, Tri10 or both genes are made constitutively active.

### Example 3

The filamentous fungus Fusarium sporotrichioides NRRL 18340 is a Tri4 mutant and accumulates trichodiene. This organism is treated to modify the Tri6 gene to have constitutive activity, thus increasing the production of FPP and further increasing trichodiene production.

### Example 4

NRRL 18340 is again modified, this time both the Tri 6 and Tri10 gene are modified such that the Tri5 gene product is increased in production. In a related example the Tri6, Tri10 or both genes are made constitutively active.

### Example 5

The NRRL 3299 is again modified, this time both the Tri 6 and Tri10 gene are modified and one or more additional copies of the Tri5 are introduced such that the Tri5 gene product is increased in production. In a related example the Tri6, Tri10 or both genes are made constitutively active.

### Example 6

The NRRL 3299 is again modified, this time using Tri6 and/or Tri10 genes from a different fungal species. Both the Tri6 and Tri10 genes are modified such that the Tri5 gene product is increased in production. In a related example the Tri6 or Tri10 or both genes are made constitutively active.

### Example 7

### Generating Expression Plasmids Encoding Tri6-PK and Tri10-P1

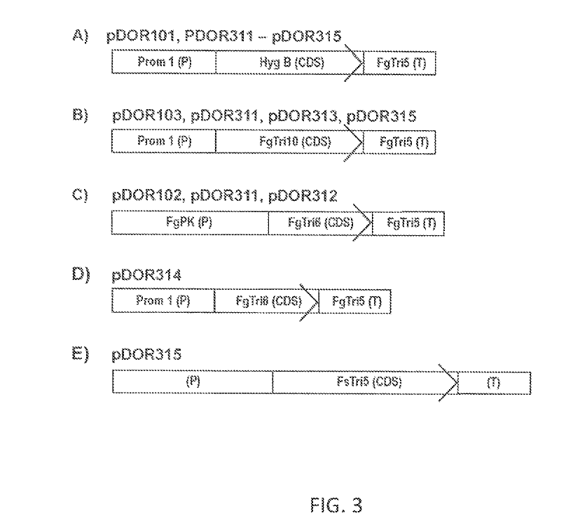

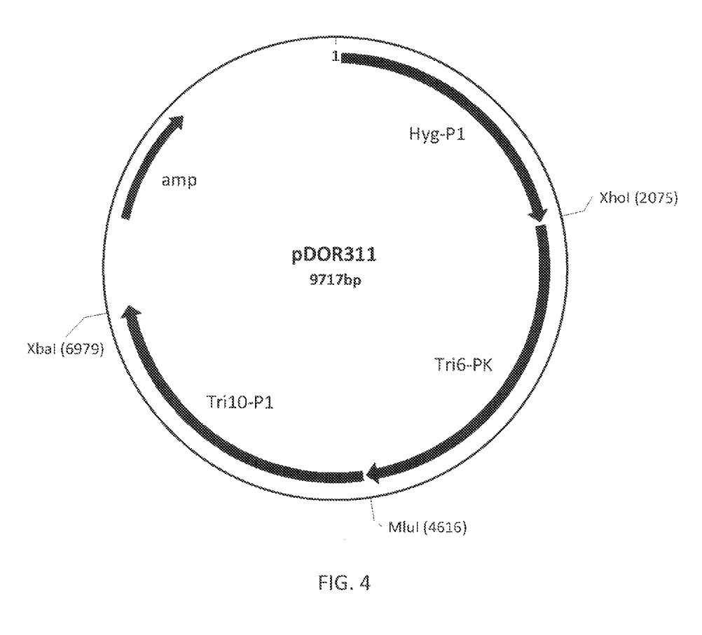

Expression plasmid pDOR311 was generated by inserting the Tri6-PK-Tri10-P1 gene fragment into the pDOR101 vector. Vector pDOR101 was generated by inserting a DNA synthesis construct comprising the Hyg-P1 (FIG. 3) gene into the EcoRV restriction site of pUC57 (GenBank accession number Y14837). Hyg-P1 consists of three genetic elements (Table 1) including hygromycin resistance selectable marker gene encoding the E. coli hygromycin phosphotransferase (GenBank accession number V01499) with the Cochliobolus heterostrophus P1 promoter sequence (GenBank accession number CCLPROA REGION: 1 . . . 645) and the Gibberella zeae Tri5 terminator sequence (GenBank accession number AF359361 REGION: 32132 . . . 32484). The Tri6-PK gene (SEQ ID NO: 1) was generated by DNA synthesis and cloned as a blunt ended fragment into the EcoRV restriction site of pUC57 to generate pDOR102. Tri6-P1 consists of the G. zeae, Tri6 coding region (GenBank accession number AF359361 REGION: 27401 . . . 28057), the G. zeae Tri5 terminator sequence, and the G. zeae pyruvate kinase promoter sequence (GenBank accession number: FG10743.1 REGION: 3790933 . . . 3792134). The Tri10-P1 gene (SEQ ID NO: 2) was generated by DNA synthesis and cloned as a blunt ended fragment into the EcoRV restriction site of pUC57 to yield pDOR103. Tri10-P1 consists of the Gibberella zeae, Tri10 coding region (GenBank accession number AF359361 REGION: 32799 . . . 34151) in which two conservative C to T nucleotide changes were introduced at positions 570 and 771 of the coding sequence designed to eliminate two consensus Tri6 DNA binding sites (YNAGGCC) proposed to function in the negative regulation of Tri10 gene expression (Tag, A. G., Garifullina, G. F., Peplow, A. W., Ake Jr., C.; Phillips, T. D., Hohn, T. M. & Beremand, M. N. (2001) A Novel Regulatory Gene, Tri10, Controls Trichothecene Toxin Production and Gene Expression, Appl. Environ. Microbiology, 67: 5294-5302), the G. zeae Tri5 terminator sequence, and the Cochliobolus heterostrophus P1 promoter sequence. To create the Tri6-PK-Tri10-P1 fragment pDOR102 DNA was digested to completion with the restriction enzymes XbaI and MluI the reaction mixture resolved by gel electrophoresis, and the 1.7 kb Tri6-PK fragment was gel extracted. The isolated fragment was ligated with pDOR103 DNA digested with restriction enzymes SpeI and MluI to generate plasmid pDOR203. The pDOR203 DNA was digested to completion with the restriction enzymes XhoI and NheI the reaction mixture resolved by gel electrophoresis, and the 4.9 kb Tri6-PK-Tri10-P1 fragment was gel extracted. The isolated fragment was ligated into XhoI XbaI digested pDOR101 yielding expression plasmid pDOR311. The nucleotide sequence of pDOR311 is given in SEQ ID NO: 3 and a plasmid map in FIG. 4.

TABLE 1
Expression Plasmid Genetic Elements

| Genetic | | | GenBank Accession |
|---|---|---|---|
| Element | Source | Function | Number |
| Promoter | C. heterostrophus | Constitutive | CCLPROA REGION: |
| 1 | | promoter | 1-645 |
| FgTri5 | F. graminearum | Tri5 transcription | AF359361 REGION: |
| term | | termination | 32132 . . . 32491 |
| FgTri6 | F. graminearum | Tri6 coding | AF359361 REGION: |
| CDS | | sequence | 27401 . . . 28057 |
| Hyg | E. coli | Hygromycin B | V01499 REGION: |
| CDS | | phosphotransferase | 231 . . . 1256 |
| | | coding sequence | |
| FgTri10 | F. graminearum | Tril0 coding | AF359361 REGION: |
| CDS | | sequence | 32799 . . . 34151 |
| FgPK | F. graminearum | Pyruvate kinase | FG10743.1 REGION: |
| prom | | promoter | 3790934 . . . 3792134 |
| FsTri5 | F. sporotrichioides | Trichodiene | AF359360 |
| gene | | synthase gene | REGION: 26809 . . . 29642 |

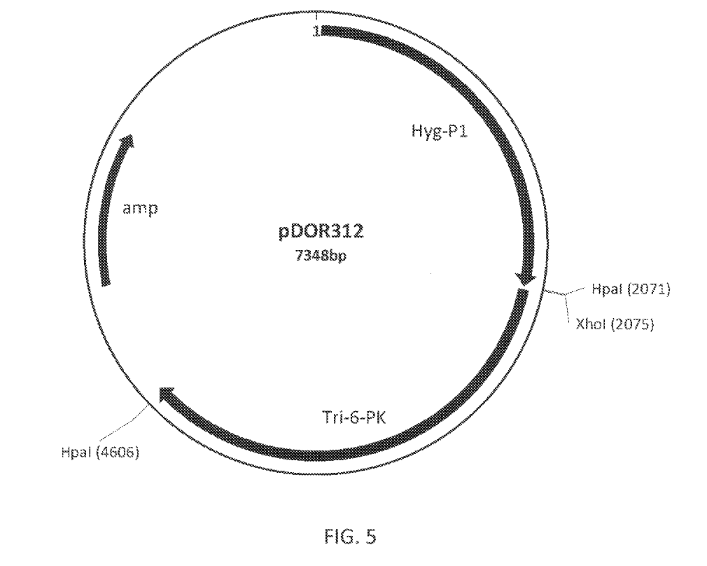

Expression plasmid pDOR312 was generated by removing the Tri10-P1 gene in pDOR311. The pDOR311 plasmid DNA was digested to completion with SpeI and XbaI restriction enzymes the reaction mixture was resolved by gel electrophoresis, and the 7.3 kb fragment was gel extracted. The isolated fragment was self-ligated yielding expression plasmid pDOR312. The nucleotide sequence of pDOR312 is given in SEQ ID NO: 4 and a plasmid map in FIG. 5.

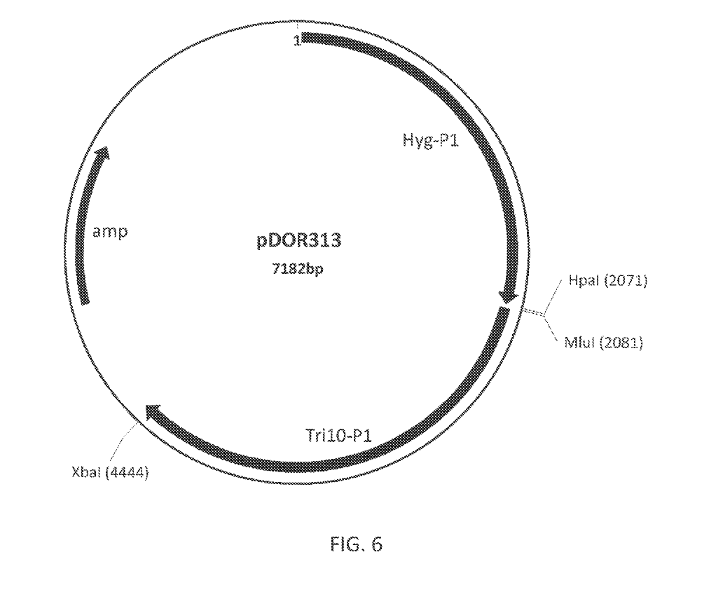

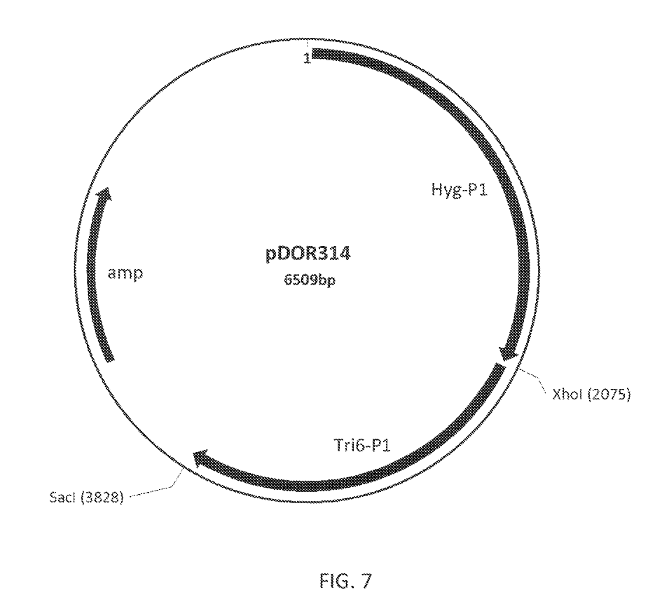

Expression plasmid pDOR313 was generated by removing the Tri6-PK gene in pDOR311. The pDOR311 plasmid DNA was digested to completion with HpaI restriction enzyme the reaction mixture was resolved by gel electrophoresis, and the 7.2 kb fragment was gel extracted. The isolated fragment was self-ligated yielding expression plasmid pDOR313. The nucleotide sequence of pDOR313 is given in SEQ ID NO: 5 and a plasmid map in FIG. 6.

Expression plasmid pDOR314 was generated by inserting the Tri6-P1 gene (SEQ ID NO: 6) into the pDOR101 vector. The Tri6-P1 gene (FIG. 3) was generated by PCR amplifying from the synthetic Tri6-PK gene in plasmid pDOR102 using primers DOR123 (SEQ ID NO: 7) and DOR107 (SEQ ID NO: 8). The upstream primer used for the amplification of the Tri6 coding sequence included a change in the codon for the second amino acid (changing an Ile residue to Val) and introduced an NcoI restriction site. The PCR product was digested to completion using NcoI and BsrGI restriction enzymes, the reaction mixture was resolved by gel electrophoresis, the 1.0 kb DNA fragment was gel extracted, and the isolated DNA fragment was ligated into the NcoI BsrGI restriction enzyme site of pDOR103 to generate plasmid pDOR202. The pDOR202 DNA was digested to completion with the restriction enzymes SpeI and SacI the reaction mixture resolved by gel electrophoresis, and the 1.7 kb Tri6-P1 fragment was gel extracted. The isolated fragment was ligated into XbaI SacI digested pDOR101 yielding expression plasmid pDOR314. The nucleotide sequence of pDOR313 is given is SEQ ID NO: 9 and a plasmid map in FIG. 7.

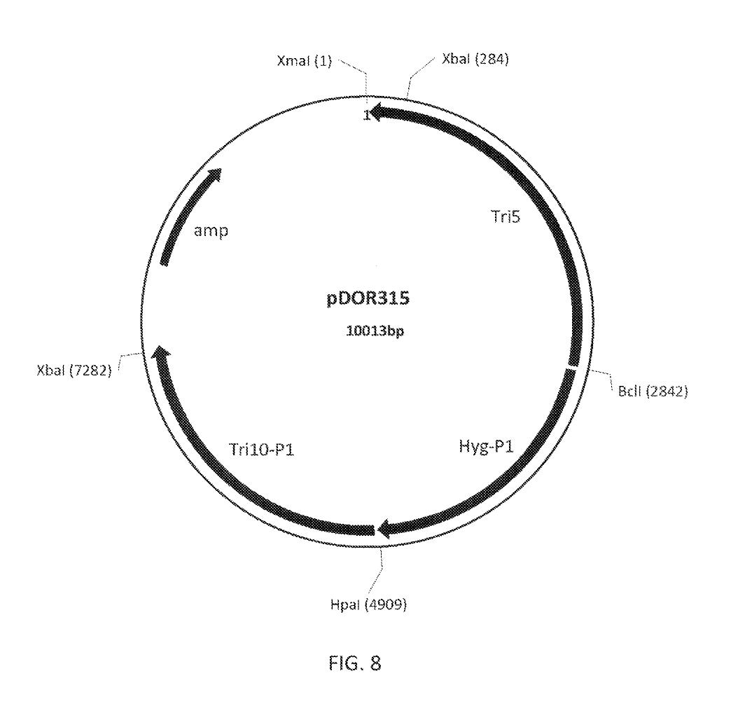

Expression plasmid pDOR315 was generated by inserting a nucleotide sequence encoding the Fusarium sporotrichioides trichodiene synthase gene (Tri5) gene into the expression plasmid pDOR312. The Tri5 gene includes the Tri5 promoter, coding sequence, and terminator sequences and its duplication would be expected to increase the expression of this key enzyme in trichodiene production. The Tri5 gene fragment was generated by PCR amplifying from Fusarium sporotrichioides T-0926 (NRRL 3299, obtained from Pennsylvania State University, Fusarium Research Center) genomic DNA the Tri5 gene (GenBank accession number AF359360 REGION:26809 . . . 29642) using primers DOR121 (SEQ ID NO: 10) and DOR122 (SEQ ID NO: 11). The upstream primer created a NheI restriction site and the downstream primer created an XmaI restriction site. The PCR product was digested to completion using NheI and XmaI restriction enzymes, the reaction mixture was resolved by gel electrophoresis, the 2.8 kb DNA fragment was gel extracted, and the isolated DNA fragment was ligated into the AvrII XmaI restriction enzyme site of expression plasmid pDOR313 yielding expression plasmid pDOR315. The nucleotide sequence of pDOR315 is given is SEQ ID NO: 12 and a plasmid map in FIG. 8.

### Example 8

This example describes the generation of Fusarium sporotrichioides host strains useful in the invention.

The host strains were created by transforming Fusarium sporotrichioides T-0927 (NRRL 18340, obtained from Pennsylvania State University, Fusarium Research Center) parent cells with one of the expression plasmids of Example 1. DNA-mediated transformations into F. sporotrichioides T-0927 protoplasts were conducted using the polyethylene glycol procedure as described by (Royer, J. C., Moyer, D. L., Reiwitch, S. G., Madden, M. S., Jensen, E. B., Brown, S. H., Yonker, C. C., Johnstone, J. A., Golightly, E. J., Yoder, W. T., and Shuster, J. R. 1995. Fusarium graminearum A 3/5 as a novel host for heterologous protein production. Nature Biotechnology 13:1479-1483). Transformed host cells were initially grown in petri plates of agar medium (0.1% casein enzyme hydrolysate, 0.1% yeast extract, 1.6% agar, and 1 M sucrose) and after 24 hours a 1% water agar overlay containing 50 μg/mL of the antibiotic hygromycin was added to select transformants that integrated the expression plasmid DNA. Single colonies growing through the overlay after 3 to 10 days were transferred to V8 juice agar (per liter: 180 mL V8 juice, 800 mL water, 2 g CaCO₃, and 15 g Bacto agar) containing hygromycin (150 μg/mL) and cultures were grown at 28 degree C. for 7 to 10 days and then conidia were harvested in sterile water. The conidia were stored at −80.degree. C. in cryo-vials in 1 mL stock aliquots made up of 200 μL sterile 50% glycerol and 800 μL suspension of conidia. All gene integrations in transformants were confirmed by phenotypic analysis and polymerase chain reaction (“PCR”) analysis of genomic DNA for DNA fragments representing the integrated genetic elements. Expression plasmids pDOR311, pDOR312, pDOR313, pDOR314, pDOR315 were constructed using the pUC57 vector and are schematically described by FIG. 4-8 and Table 1. Propagation of plasmid DNA was performed in Escherichia coli strain DH5α.

### Example 9

This example demonstrates increased production of trichodiene in parent strain Fusarium sporotrichioides T-0926 as compared to host strain Fusarium sporotrichioides T-0927.

Fusarium sporotrichioides T-0927 is a UV mutant strain (Tri4⁻) derived from isolate F. sporotrichioides T-0926 that is blocked in the Tri4 step of the trichothecene pathway and accumulates trichodiene. Inoculum cultures of F. sporotrichioides strains T-0926 and strain T-0927 were established on V8 agar medium. After 7 days conidia were harvested using cell scrapers and used to inoculate at an initial number of 1×10⁵ spores/mL in separate 250 mL flasks containing 45 mL of GYEP medium (0.1% Bacto yeast extract, 0.1% Bacto peptone, and 5% glucose). Cultures were incubated at 28 degree. C. on a rotary shaker at 200 RPM for 24 hours at which point they were overlain with 5 mL of dodecane. At 48 hours 0.45 ml of YEP medium (5% Bacto peptone and 1% Bacto yeast extract) was added and after 120 hours culture material was transferred to a 50 mL centrifuge tube and centrifuged for 5 min at 5000×g after which samples of the organic overlay layer were taken. Dry weight of the fungal mycelium was determined by filtration of culture material on pre-dried and pre-weighed filters which were dried at 80 degree. C. for 3 days and weighed to generate the culture dry weight (CDW).

A volume of 4 μL of the organic overlay sample was added to a clean glass vial containing 996 μL of isopropyl alcohol with beta- or trans-caryophyllene (Sigma-Aldrich, St. Louis, Mo.) as an internal standard prior to analysis. Samples were analyzed on a Hewlett-Packard 6890 gas chromatograph (GC) coupled to a 5973 mass selective detector (MSD) outfitted with a 7683 series injector and autosampler and equipped with an Zebron ZB-Wax plus wax capillary column (0.25 mm i.d.×30 m with 0.25 mm film) (available from Agilent Technologies). For all experiments, needle sampling depth was set to 8 mm. The GC was operated at a He flow rate of 2 mL min¹, and the MSD operated at 70 eV. Splitless injections (2 μL) were performed with an injector temperature of 250° C. The GC was programmed with an initial oven temperature of 50° C. (5-min hold), which is then increased 10° C. min¹ up to 180° C. (4-min hold), followed by a 100° C. min¹ ramp until 240° C. (1-min hold). A solvent delay of 8.5 min was included prior to the acquisition of MS data. Product peaks are quantified by integration of peak areas using Enhanced Chemstation (version B.01.00, Agilent Technologies). Trichodiene was identified based on its published trichodiene mass fragmentation profile (Desjardins A E, Plattner R D & Beremand M N. (1987) Ancymidol blocks trichothecene biosynthesis and leads to accumulation of trichodiene in Fusarium sporotrichioides and Gibberella pulicaris. Appl. Environ. Microbiol., 53:1860-1865) and had a retention time of 18.48 minutes using this GC protocol. Caryophyllene was used as a standard for quantitation and had a retention time of 15.92 minutes. A response factor was established for caryophyllene based on the GC peak area/mg/mL where a caryophyllene peak area corresponding to a concentration of 1.0 mg/mL equals 1.0 CP unit. Trichodiene titer was calculated as the ratio of the peak area for trichodiene to the peak area of the caryophyllene response factor and reported in CP units.

After 120 hours of growth, two Fusarium sporotrichioides T-0927 cultures were found to produce 11 and 17 CP units trichodiene/g CDW and two Fusarium sporotrichioides T-0926 cultures were both found to produce 0.00 CP units trichodiene/g CDW.

### Example 10

This example demonstrates increased production of trichodiene in host strains expressing both Tri6-PK and Tri10-P1 as compared to production by the parent strain Fusarium sporotrichioides T-0927.

Inoculum cultures of host strains B01 and B07 (Table 2) were established by growing a stock aliquot of each strain on V8 agar medium with hygromycin (150 μg/mL) for 7 to 10 days. Conidia were harvested from inoculum cultures using cell scrapers and used to inoculate at an initial number of 1×10⁵ spores/mL in separate 250 mL flasks containing 45 mL of GYEP medium (0.1% Bacto yeast extract, 0.1% Bacto peptone, and 5% glucose). Cultures were incubated at 28 degree. C. on a rotary shaker at 200 RPM for 24 hours at which point they were overlain with 5.0 mL of dodecane. At 48 hours 0.45 mL of YEP medium (5% Bacto peptone and 1% Bacto yeast extract) was added and after 120 hours culture material was transferred to a 50 mL centrifuge tube and centrifuged for 5 min at 5000×g after which samples of the organic overlay layer were taken for analysis. Dry weight of the fungal mycelium was determined by filtration of culture material on pre-dried and pre-weighed filters which were dried at 80 degree. C. for 3 days and weighed to generate the culture dry weight (CDW).

A volume of 4 μL of the organic overlay sample was added to 996 μL of isopropyl alcohol containing caryophyllene as an internal standard in a clean glass vial prior to analysis. The diluted organic overlay samples were analyzed on a Hewlett-Packard 6890 gas chromatograph/mass spectrometer (GC/MS) as described in Example 3. Experiments were performed using 2 replicates of each host strain, and results were averaged.

After 120 hours of growth, host strains B01 and B07 were found to produce 111 CP units trichodiene/gCDW trichodiene and 103 CP units trichodiene/gCDW, Parent strain Fusarium sporotrichioides T-0927 cultures were found to produce 14 CP units trichodiene g CDW.

TABLE 2
Host strain Plasmid Source

| | | | Fungal |
|---|---|---|---|
| Host | | Expression | Antibiotic |
| strain | Parent Strain | Plasmids | Selection |
| B01 | Fusarium sporotrichioides T-0927 | pDOR311 | Hygromycin |
| B07 | Fusarium sporotrichioides T-0927 | pDOR311 | Hygromycin |
| G08 | Fusarium sporotrichioides T-0927 | pDOR312 | Hygromycin |
| H03 | Fusarium sporotrichioides T-0927 | pDOR313 | Hygromycin |
| H07 | Fusarium sporotrichioides T-0927 | pDOR313 | Hygromycin |
| J01 | Fusarium sporotrichioides T-0927 | pDOR314 | Hygromycin |
| J10 | Fusarium sporotrichioides T-0927 | pDOR314 | Hygromycin |
| I01 | Fusarium sporotrichioides T-0927 | pDOR315 | Hygromycin |

### Example 11

This example demonstrates increased production of trichodiene in host strains expressing Tri6-PK as compared to production by the parent strain Fusarium sporotrichioides T-0927.

Inoculum cultures of host strain G08 was established by growing a stock aliquot of each strain on V8 agar medium with hygromycin (150 μg/mL) for 7 to 10 days. Conidia were harvested from inoculum cultures using cell scrapers and used to inoculate at an initial number of 1×10⁵ spores/mL in separate 125 mL flasks containing 62.5 mL of GYEP medium (0.1% Bacto yeast extract, 0.1% Bacto peptone, and 5% glucose). Cultures were incubated at 28 degree. C. on a rotary shaker at 200 RPM for 24 hours at which point they were overlain with 6.25 mL of dodecane. After 168 hours 45 mL of culture material enriched for the organic layer was transferred to a 50 mL centrifuge tube and centrifuged for 5 min at 5000×g after which samples of the organic overlay layer were taken. Dry weight of the fungal mycelium was determined by filtration of culture material on pre-dried and pre-weighed filters which were dried at 80 degree. C. for 3 days and weighed to generate the culture dry weight (CDW).

A volume of 4 μl of the organic overlay sample was added to 996 μl of isopropyl alcohol containing caryophyllene as an internal standard in a clean glass vial prior to analysis. The diluted organic overlay samples were analyzed on a Hewlett-Packard 6890 gas chromatograph/mass spectrometer (GC/MS) as described in Example 3.

After 168 hours of growth, host strain G08 was found to produce 268 CP units trichodiene/gCDW, a parent strain Fusarium sporotrichioides T-0927 culture was found to produce 27 CP units trichodiene/gCDW

### Example 12

This example demonstrates increased production of trichodiene in host strains expressing Tri10-P1 as compared to production by the parent strain Fusarium sporotrichioides T-0927.

Inoculum cultures of host strains H03 and H07 were established by growing a stock aliquot of each strain on V8 agar medium with hygromycin (150 μg/mL) for 7 to 10 days. Conidia were harvested from inoculum cultures using cell scrapers and used to inoculate at an initial number of 1×10⁵ spores/mL in separate 125 mL flasks containing 62.5 mL of GYEP medium (0.1% Bacto yeast extract, 0.1% Bacto peptone, and 5% glucose). Cultures were incubated at 28 degree. C. on a rotary shaker at 200 RPM for 24 hours at which point they were overlain with 6.25 mL of dodecane. After 168 hours 45 mL of culture material enriched for the organic layer was transferred to a 50 mL centrifuge tube and centrifuged for 5 min at 5000×g after which samples of the organic overlay layer were taken for analysis. Dry weight of the fungal mycelium was determined by filtration of culture material on pre-dried and pre-weighed filters which were dried at 80 degree. C. for 3 days and weighed to generate the culture dry weight (CDW).

A volume of 4 μL of the organic overlay sample was added to 996 μL of isopropyl alcohol containing caryophyllene as an internal standard in a clean glass vial prior to analysis. The diluted organic overlay samples were analyzed on a Hewlett-Packard 6890 gas chromatograph/mass spectrometer (GC/MS) as described in Example 3.

After 168 hours of growth, host strains H03 and H07 were found to produce 143 CP units trichodiene/gCDW and 150 CP units trichodiene/gDCW, a Fusarium sporotrichioides T-0927 culture was found to produce 27 CP units trichodiene/gDCW.

### Example 13

This example demonstrates increased production of trichodiene in host strains expressing both Tri10-P1 and a plurality of Tri5 as compared to production by the parent strain Fusarium sporotrichioides T-0927.

Inoculum cultures of host strains J01 and J10 were established by growing a stock aliquot of each strain on V8 agar medium with hygromycin (150 μg/mL) for 7 to 10 days. Conidia were harvested from inoculum cultures using cell scrapers and used to inoculate at an initial number of 1×10⁵ spores/mL in separate 250 mL flasks containing 45 mL of GYEP medium (0.1% Bacto yeast extract, 0.1% Bacto peptone, and 5% glucose). Cultures were incubated at 28 degree. C. on a rotary shaker at 200 RPM for 24 hours at which point they were overlain with 5 mL of dodecane. At 48 hours 0.45 ml of YEP medium (5% Bacto peptone and 1% Bacto yeast extract) was added and after 120 hours culture material was transferred to a 50 mL centrifuge tube and centrifuged for 5 min at 5000×g after which samples of the organic overlay layer were taken for analysis. Dry weight of the fungal mycelium was determined by filtration of culture material on pre-dried and pre-weighed filters which were dried at 80 degree. C. for 3 days and weighed to generate the culture dry weight (CDW).

A volume of 4 μL of the organic overlay sample was added to 996 μL of isopropyl alcohol containing caryophyllene as an internal standard in a clean glass vial prior to analysis. The diluted organic overlay samples were analyzed on a Hewlett-Packard 6890 gas chromatograph/mass spectrometer (GC/MS) as described in Example 3. Experiments were performed using 2 replicates of each host strain, and results were averaged.

After 120 hours of growth, host strains J01 and J10 were found to produce 141 CP units trichodiene/gCDW and 151 CP units trichodiene/gCDW, parent strain Fusarium sporotrichioides T-0927 cultures were found to produce 14 CP units trichodiene/gCDW.

### Example 14

This example demonstrates increased production of trichodiene in host strains expressing Tri6-P1 as compared to production by the parent strain Fusarium sporotrichioides T-0927.

Inoculum cultures of host strain 101 was established by growing a stock aliquot of each strain on V8 agar medium with hygromycin (150 μg/mL) for 7 to 10 days. Conidia were harvested from inoculum cultures using cell scrapers and used to inoculate at an initial number of 1×10⁵ spores/mL in separate 125 mL flasks containing 62.5 mL of GYEP medium (0.1% Bacto yeast extract, 0.1% Bacto peptone, and 5% glucose). Cultures were incubated at 28 degree. C. on a rotary shaker at 200 RPM for 24 hours at which point they were overlain with 6.25 mL of dodecane. After 168 hours 45 mL of culture material enriched for the organic layer was transferred to a 50 mL centrifuge tube and centrifuged for 5 min at 5000×g after which samples of the organic overlay layer were taken. Dry weight of the fungal mycelium was determined by filtration of culture material on pre-dried and pre-weighed filters which were dried at 80 degree. C. for 3 days and weighed to generate the culture dry weight (CDW).

A volume of 4 μL of the organic overlay sample was added to 996 μL of isopropyl alcohol containing caryophyllene as an internal standard in a clean glass vial prior to analysis. The diluted organic overlay samples were analyzed on a Hewlett-Packard 6890 gas chromatograph/mass spectrometer (GC/MS) as described in Example 3.

After 168 hours of growth, host strain I01 was found to produce 117 CP units trichodiene/gCDW, a parent strain Fusarium sporotrichioides T-0927 culture was found to produce 27 CP units trichodiene/gCDW.

1-15. (canceled)

16. A method of producing trichodiene comprising:
a) selecting a mutant filamentous fungus having the trichothecene pathway comprising:
i. a disrupted Tri4 gene, a mutant Tri4 gene having low P450 monooxygenase production, and/or the presence of a Tri4 inhibitor sufficient to inhibit at least a portion of the Tri4 gene product;
ii. a modified gene selected from Tri5, Tri6 and Tri10, the gene modified to increase the production of the gene product;
b) cultivating the mutant filamentous fungus in a growth media culture; and
c) isolating trichodiene from the growth media culture;
wherein the mutant filamentous fungus produces at least 10% more trichodiene than the parent filamentous fungus when cultured under the same conditions.

17-18. (canceled)

19. The method according to claim 16 wherein the mutant filamentous fungus produces at least 0.25 g of trichodiene per gram of glucose or glucose equivalent consumed.

20. (canceled)

21. The method according to claim 16 wherein the mutant filamentous fungus is selected from the group consisting of Acremonium, Aspergillus, Aureobasidium, Cryptococcus, Filibasidium, Fusarium, Gibberella, Humicola, Magnaporthe, Mucor, Myceliophthora, Myrothecium, Neocallimastix, Neurospora, Paecilomyces, Penicillium, Piromyces, Stachybotrys, Schizophyllum, Talaromyces, Thermoascus, Thielavia, Tolypocladium, Trichoderma, or Trichothecium strain.

22. The method according to claim 21 wherein the mutant filamentous fungus is Fusarium sporotrichioides.

23-25. (canceled)

26. The method according to claim 16 wherein the mutant filamentous fungus comprises a disrupted Tri4 gene, or a mutant Tri4 gene having low P450 monooxygenase production.

27. The method according to claim 16 wherein the mutant filamentous fungus comprises a nonrevertable site-selected deletion of part or all of nucleic acid encoding the Tri4 gene product such that the Tri4 gene is inactivated.

28. The method according to claim 16 wherein the Tri4 gene product enzymatic or catalytic activity is reduced by at least 10% when compared to the parent strain under the same conditions.

29. The method according to claim 16 wherein the mutant filamentous fungus is in the presence of a Tri4 inhibitor sufficient to inhibit at least a portion of the Tri4 gene product.

30. The method according to claim 16 wherein the modified gene has been modified to have constitutive activity in producing the gene product.

31. The method according to claim 16 wherein the mutant filamentous fungus comprises a modified Tri5 gene, the gene modified to increase the production of the gene product.

32. The method according to claim 16 wherein the mutant filamentous fungus comprises a modified Tri6 gene, the gene modified to increase the production of the gene product.

33. The method according to claim 16 wherein the mutant filamentous fungus comprises a modified Tri10 gene, the gene modified to increase the production of the gene product.

34. The method according to claim 16 wherein the mutant filamentous fungus comprises at least two modified genes selected from Tri5, Tri6, and Tri10, the genes modified to increase the production of the gene products.

35. The method according to claim 16 wherein the mutant filamentous fungus comprises a modified Tri5 gene, a modified Tri6 gene, and a modified Tri10 gene, the genes modified to increase the production of the gene products.

36. The method according to claim 16 wherein the modified gene comprises more than one copy of the nucleic acid sequence encoding for the gene product.

37. The method according to claim 36 wherein at least one of the additional copies of the nucleic acid sequence encoding for the gene product is in a vector which is capable of autonomous maintenance in the filamentous fungus.

38. The method according to claim 16 wherein the modified gene comprises a coding sequence operably linked to a promoter from a constitutively active filamentous fungal gene.

39. The method according to claim 16 wherein the mutant filamentous fungus is Fusarium.

40. The method according to claim 16 wherein the mutant filamentous fungus is Fusarium venenatam.

41. The method according to claim 16 wherein the mutant filamentous fungus is Fusarium gramineareum.

42. The method according to claim 16 wherein the growth media comprises growth media prepared from biomass.

43. The method according to claim 16 wherein the growth media comprises growth media prepared from lignocellulosic feedstock.


SEQUENCE LISTING (12 sequences)

SEQ ID NO: 1 - DNA, 2561 bp, Artificial sequence
  Description of artificial sequence Gibberella zeae 26-1527 Gibberella zeae 1528-2183 Gibberella zeae 2184-2543 (Gibberella zeae 26-2543)
gggagctggttaacctcgagaccggtagcttggactgtaacagacggaagtcgggaagccatgataataatagtaaacaagtttctatttggagatttgtttgaaacgtgataacaaagctataacctttaataattatagactttttgtttctaaatttggacaattctagggtttgttacatgtatctgactttgatcttgcacgggaagcggggtggcagagctccccataattctgaaccagatcgccagaacgagagaagaaaaaaaagaaaaaagtaaaaaaaagagctcctcctatggatctcaaccacgggcaaaaacaaagctcagactgtgtaatccatgctaagtacatgtgaggcaagtactccgtagcgctactcctggcttggctccctacccctccagtcatgatggctgcaaatttcttgggggaggtacacatgtttgttgattggggaggaatgagtaaagtaccctgggcttgaatgaaagcggtaggaggtaggtagagggaggaatttagccttgccttgtcttggccgtaggtttgctgtaatattttttgtctttattatcatcaccacaccaactctacttccttctcctctcctcttcctttctctctctctcttcctcatctctcaacctttcttgaccttcgacgtcaaaacagctttacctccactcacccctctacgcctcgacctttgcgagccccccctccttcgtcgaacgtataatactaccaggaagagcgcacgttcctctgcctcttctgatcatatcatccctttggattattatcgtacggtgcaacactcgtacaaagatatccatatcccctttgtaagtcgacatgctaccgttgcttctgtgctgcacctagatggagacggttggtgtggcttgggccctgagatcccgctcttctgcgcttctctggcaatggagatagggaaatatgtggcaattccggttttggcagatgcatttctggtcttgtttccggacctgcttcagaatgtcgtttgttttcgacccagaaattacgaaccgccggatttgttctcggttcttctggcccgattagcgacaagtctgacgattcttaactcggcccatctattgagctcatgggtcatcctgtgtccttctcactagccaaaccagccgttttgcgtcgcgccgaatgctctgcccaccaaattaccacgccccatccctatcgcctttcacaccacgtcacttaacgtcagatcccacgttagccccccatgccttttctatacctcacgaaagagtctccgttggcctgcataacaagtgtccgcagaacagcctgatatccatccctgttatcagtagtgggatctacaatcgttcgatcaattgagatatatatgtgtgctggttgtccaatctgcaccacacctgttgtctcatcatcttcattcctttcgcagccagagctaccccgccaagctcttctctcacaaacaacttgctaaccacattcacaatgatttacatggaggccgaatctcactacgaatcttggagcgccttgcccctctttgatcgagttgcgtctcccgatcctgccaaggactttgtcccagatctaaacgactatgaatcaccaacattcgaaatagatcttctctcagaaacttatgactttgacaacttccccacatactctctaccaacggtggattcaaccaagactttgtactccgaagaaccacttgtttgcttcgactttgacttcgcgaacccggctatcgaaaattatataaccacatcgtcgggactgttggacgcagtgccaagccagcttatcgcccttcccaccttcacacggccaagcaaatgcccattccctagttgcaagtcggccacagtctttgaaagcggacgggactttaggcggcattaccggcaacacttcaagcgctttttctgtcgctactcagaatgccctcagtcagctcaagacctgcaagaagtcggcaccaaaggctttgcgactcgcaaggaccgtgctcggcatgagtctaagcacaaaccaacagtgcggtgcccttggcaagacaaggaaggacaacaatgtctgagggtctttagcagggtggataacatgcgagatcactataggcggatacataagtgttgaccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggagggagctggttaacactagtacgcgt

SEQ ID NO: 2 - DNA, 2428 bp, Artificial sequence
  Description of artificial sequence Cochliobolus heterostrophus 26-670 Gibberella zeae 671-2029 Gibberella zeae 2030-2383 (Gibberella zeae 671-2383)
gggagctggttaacactagtacgcgtgtcgacgccgctccaccggctgcacatgtcaaggcccacctgcgcattgccatatgaaaatggcctgacatcatgttgaccggcccagcaaacctctgcagagcggtcaagctggcggcgagctttgtagctcgaccacgatgggaatgggaatgcagcagcccagagccatgtgtgccctggtcccccggctcaagtaagccgtggttgtggggatgaaccatccaccgcagcaagttttcttccccattgccctcgtcggccttctgtgagcttctaacacatctgccgcctctgttgactccctctcctcctcccagaccctctggtttgccctcctgtgacctgcacaatctgccccgcgccagtgtcatagtccttggtacgtccccaccaccgcgcgcctccctctctctgtccgctatctcgaccatatcaccaccacagagatagcaccagagtgacgtctcgtgccctgcagcgtgcctcgtgtcgtctgtcttgtcaaccgtcttctcctgcctgccaataccccataccacctcaaatgacccagtcgcaacccttctgcccttaccagccctcggagcttatcgatatctaaccctttgatatagtgcaacaggtgctcagcctacaatcgccatggatttcccaaagcctagacaagtccgagagacgagcctgttgatgtactacctagacgtcgtgtttcctctgcaatgcatcaacccaaacaacaattgtctgggaaagagagagtggctgttgactatactgacctctgcgcggcctacgtactatgccacattgtgcatgtcgctcctctataaagaatcgctttcaagcccttgcagatctgaacaggcgatggtatggaagagagagaagacatactactacattcttgcactccaggagtctcagaagctgctgggtgggctcgacaagacatttggcatcacaaggctgaaaggtaccgtcgttgcccttgcttgcatgctacagcttatcagttttgaggtaagacgaatccaccattgtttcgatgctcgatgtcgatgctcgatatccgatctacgattatcgttggtcactaacaaattaaaatagtcttcgcacctaagcaggggagattggcgcgttcacctccatgcggccaacatactcattcctgtcttggttgagggatggtccacagctttgcaatcaggtcccccagccacctccatatggtgcgagctggatgaatcacacttcggctcgactgaagatcaaacctctttgagcttcgaatacgtcggagctttgagattcctgtcaaactcactcgccgcagtcggcatcctgtcttgcatatctattggcccatcagcaccatttgaagattacggccatctcctggaccagccaggtcttatacagctggacgaggtgctggggtgcaggaattggaccatgttgactattctcgaagtgggtaagctggatcgttggaagcgacaggagcaagaacataatcgcttgagcctaaagacgctcgctaggcgcgccatgatgattgaggatatgttgtcagacgagctacaaaggctaccgacagacgagacgcttccagacctcatcactcagatttacgccgcctctatcatgacgtatctgcatacagtagtttccggactcaatcccaacctttcagaggttcaggatagtgtggccgggacgcttcaattgttggagaggctcccaaatcttgaagctgtcacgagcgttacttggcctctagctgtcacaggatgcatggcctcagaaagtcataaggactttttcagaaatactctgaggtcgtatgaggcgacattcagctccttaaaaaagtatgacggaactcttcaggtcttggaagacgcttggaagagaagagagatagatacagagtctccaatgagatgggaagacttgacggatcaccatgggcttccagtgctactttggtagggatggccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggaggtctagattggccaatagcggccgcattgtggctagcagtttcatg

SEQ ID NO: 3 - DNA, 9717 bp, Artificial sequence
  Description of artificial sequence Cochliobolus heterostrophus 45-683 Escherichia coli 684-1708 Gibberella zeae 1709-2068 Gibberella zeae 2087-3586 Gibberella zeae 3587-4244 Gibberella zeae 4245-4603 (Gibberella zeae 2087-4603) Cochliobolus heterostrophus 4622-5266 Gibberella zeae 5267-6625 Gibberella zeae 6626-6978 (Gibberella zeae 5267-6978) Cloning vector pUC57 7017-9717
cgacagagtcctaggagttgcgttaattaagccgcatttggccagccgctccaccggctgcacatgtcaaggcccacctgcgcattgccatatgaaaatggcctgacatcatgttgaccggcccagcaaacctctgcagagcggtcaagctggcggcgagctttgtagctcgaccacgatgggaatgggaatgcagcagcccagagccatgtgtgccctggtcccccggctcaagtaagccgtggttgtggggatgaaccatccaccgcagcaagttttcttccccattgccctcgtcggccttctgtgagcttctaacacatctgccgcctctgttgactccctctcctcctcccagaccctctggtttgccctcctgtgacctgcacaatctgccccgcgccagtgtcatagtccttggtacgtccccaccaccgcgcgcctccctctctctgtccgctatctcgaccatatcaccaccacagagatagcaccagagtgacgtctcgtgccctgcagcgtgcctcgtgtcgtctgtcttgtcaaccgtcttctcctgcctgccaataccccataccacctcaaatgacccagtcgcaacccttctgcccttaccagccctcggagcttatcgatatctaaccctttgatatagtgcaacaggtgctcagcctacaatcgccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggagggagctggttaacctcgagaccggtagcttggactgtaacagacggaagtcgggaagccatgataataatagtaaacaagtttctatttggagatttgtttgaaacgtgataacaaagctataacctttaataattatagactttttgtttctaaatttggacaattctagggtttgttacatgtatctgactttgatcttgcacgggaagcggggtggcagagctccccataattctgaaccagatcgccagaacgagagaagaaaaaaaagaaaaaagtaaaaaaaagagctcctcctatggatctcaaccacgggcaaaaacaaagctcagactgtgtaatccatgctaagtacatgtgaggcaagtactccgtagcgctactcctggcttggctccctacccctccagtcatgatggctgcaaatttcttgggggaggtacacatgtttgttgattggggaggaatgagtaaagtaccctgggcttgaatgaaagcggtaggaggtaggtagagggaggaatttagccttgccttgtcttggccgtaggtttgctgtaatattttttgtctttattatcatcaccacaccaactctacttccttctcctctcctcttcctttctctctctctcttcctcatctctcaacctttcttgaccttcgacgtcaaaacagctttacctccactcacccctctacgcctcgacctttgcgagccccccctccttcgtcgaacgtataatactaccaggaagagcgcacgttcctctgcctcttctgatcatatcatccctttggattattatcgtacggtgcaacactcgtacaaagatatccatatcccctttgtaagtcgacatgctaccgttgcttctgtgctgcacctagatggagacggttggtgtggcttgggccctgagatcccgctcttctgcgcttctctggcaatggagatagggaaatatgtggcaattccggttttggcagatgcatttctggtcttgtttccggacctgcttcagaatgtcgtttgttttcgacccagaaattacgaaccgccggatttgttctcggttcttctggcccgattagcgacaagtctgacgattcttaactcggcccatctattgagctcatgggtcatcctgtgtccttctcactagccaaaccagccgttttgcgtcgcgccgaatgctctgcccaccaaattaccacgccccatccctatcgcctttcacaccacgtcacttaacgtcagatcccacgttagccccccatgccttttctatacctcacgaaagagtctccgttggcctgcataacaagtgtccgcagaacagcctgatatccatccctgttatcagtagtgggatctacaatcgttcgatcaattgagatatatatgtgtgctggttgtccaatctgcaccacacctgttgtctcatcatcttcattcctttcgcagccagagctaccccgccaagctcttctctcacaaacaacttgctaaccacattcacaatgatttacatggaggccgaatctcactacgaatcttggagcgccttgcccctctttgatcgagttgcgtctcccgatcctgccaaggactttgtcccagatctaaacgactatgaatcaccaacattcgaaatagatcttctctcagaaacttatgactttgacaacttccccacatactctctaccaacggtggattcaaccaagactttgtactccgaagaaccacttgtttgcttcgactttgacttcgcgaacccggctatcgaaaattatataaccacatcgtcgggactgttggacgcagtgccaagccagcttatcgcccttcccaccttcacacggccaagcaaatgcccattccctagttgcaagtcggccacagtctttgaaagcggacgggactttaggcggcattaccggcaacacttcaagcgctttttctgtcgctactcagaatgccctcagtcagctcaagacctgcaagaagtcggcaccaaaggctttgcgactcgcaaggaccgtgctcggcatgagtctaagcacaaaccaacagtgcggtgcccttggcaagacaaggaaggacaacaatgtctgagggtctttagcagggtggataacatgcgagatcactataggcggatacataagtgttgaccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggagggagctggttaacactagtacgcgtgtcgacgccgctccaccggctgcacatgtcaaggcccacctgcgcattgccatatgaaaatggcctgacatcatgttgaccggcccagcaaacctctgcagagcggtcaagctggcggcgagctttgtagctcgaccacgatgggaatgggaatgcagcagcccagagccatgtgtgccctggtcccccggctcaagtaagccgtggttgtggggatgaaccatccaccgcagcaagttttcttccccattgccctcgtcggccttctgtgagcttctaacacatctgccgcctctgttgactccctctcctcctcccagaccctctggtttgccctcctgtgacctgcacaatctgccccgcgccagtgtcatagtccttggtacgtccccaccaccgcgcgcctccctctctctgtccgctatctcgaccatatcaccaccacagagatagcaccagagtgacgtctcgtgccctgcagcgtgcctcgtgtcgtctgtcttgtcaaccgtcttctcctgcctgccaataccccataccacctcaaatgacccagtcgcaacccttctgcccttaccagccctcggagcttatcgatatctaaccctttgatatagtgcaacaggtgctcagcctacaatcgccatggatttcccaaagcctagacaagtccgagagacgagcctgttgatgtactacctagacgtcgtgtttcctctgcaatgcatcaacccaaacaacaattgtctgggaaagagagagtggctgttgactatactgacctctgcgcggcctacgtactatgccacattgtgcatgtcgctcctctataaagaatcgctttcaagcccttgcagatctgaacaggcgatggtatggaagagagagaagacatactactacattcttgcactccaggagtctcagaagctgctgggtgggctcgacaagacatttggcatcacaaggctgaaaggtaccgtcgttgcccttgcttgcatgctacagcttatcagttttgaggtaagacgaatccaccattgtttcgatgctcgatgtcgatgctcgatatccgatctacgattatcgttggtcactaacaaattaaaatagtcttcgcacctaagcaggggagattggcgcgttcacctccatgcggccaacatactcattcctgtcttggttgagggatggtccacagctttgcaatcaggtcccccagccacctccatatggtgcgagctggatgaatcacacttcggctcgactgaagatcaaacctctttgagcttcgaatacgtcggagctttgagattcctgtcaaactcactcgccgcagtcggcatcctgtcttgcatatctattggcccatcagcaccatttgaagattacggccatctcctggaccagccaggtcttatacagctggacgaggtgctggggtgcaggaattggaccatgttgactattctcgaagtgggtaagctggatcgttggaagcgacaggagcaagaacataatcgcttgagcctaaagacgctcgctaggcgcgccatgatgattgaggatatgttgtcagacgagctacaaaggctaccgacagacgagacgcttccagacctcatcactcagatttacgccgcctctatcatgacgtatctgcatacagtagtttccggactcaatcccaacctttcagaggttcaggatagtgtggccgggacgcttcaattgttggagaggctcccaaatcttgaagctgtcacgagcgttacttggcctctagctgtcacaggatgcatggcctcagaaagtcataaggactttttcagaaatactctgaggtcgtatgaggcgacattcagctccttaaaaaagtatgacggaactcttcaggtcttggaagacgcttggaagagaagagagatagatacagagtctccaatgagatgggaagacttgacggatcaccatgggcttccagtgctactttggtagggatggccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggaggtctagattggccaatagcggccgcattgtggctagatgcattcgcgaggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaggcctctgcagtcgacgggcccgggatccgat

SEQ ID NO: 4 - DNA, 7348 bp, Artificial sequence
  Description of artificial sequence Cochliobolus heterostrophus 45-683 Escherichia coli 684-1708 Gibberella zeae 1709-2068 Gibberella zeae 2087-3586 Gibberella zeae 3587-4244 Gibberella zeae 4245-4603 (Gibberella zeae 2087-4603) Cloning vector pUC57 4648-7348
cgacagagtcctaggagttgcgttaattaagccgcatttggccagccgctccaccggctgcacatgtcaaggcccacctgcgcattgccatatgaaaatggcctgacatcatgttgaccggcccagcaaacctctgcagagcggtcaagctggcggcgagctttgtagctcgaccacgatgggaatgggaatgcagcagcccagagccatgtgtgccctggtcccccggctcaagtaagccgtggttgtggggatgaaccatccaccgcagcaagttttcttccccattgccctcgtcggccttctgtgagcttctaacacatctgccgcctctgttgactccctctcctcctcccagaccctctggtttgccctcctgtgacctgcacaatctgccccgcgccagtgtcatagtccttggtacgtccccaccaccgcgcgcctccctctctctgtccgctatctcgaccatatcaccaccacagagatagcaccagagtgacgtctcgtgccctgcagcgtgcctcgtgtcgtctgtcttgtcaaccgtcttctcctgcctgccaataccccataccacctcaaatgacccagtcgcaacccttctgcccttaccagccctcggagcttatcgatatctaaccctttgatatagtgcaacaggtgctcagcctacaatcgccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggagggagctggttaacctcgagaccggtagcttggactgtaacagacggaagtcgggaagccatgataataatagtaaacaagtttctatttggagatttgtttgaaacgtgataacaaagctataacctttaataattatagactttttgtttctaaatttggacaattctagggtttgttacatgtatctgactttgatcttgcacgggaagcggggtggcagagctccccataattctgaaccagatcgccagaacgagagaagaaaaaaaagaaaaaagtaaaaaaaagagctcctcctatggatctcaaccacgggcaaaaacaaagctcagactgtgtaatccatgctaagtacatgtgaggcaagtactccgtagcgctactcctggcttggctccctacccctccagtcatgatggctgcaaatttcttgggggaggtacacatgtttgttgattggggaggaatgagtaaagtaccctgggcttgaatgaaagcggtaggaggtaggtagagggaggaatttagccttgccttgtcttggccgtaggtttgctgtaatattttttgtctttattatcatcaccacaccaactctacttccttctcctctcctcttcctttctctctctctcttcctcatctctcaacctttcttgaccttcgacgtcaaaacagctttacctccactcacccctctacgcctcgacctttgcgagccccccctccttcgtcgaacgtataatactaccaggaagagcgcacgttcctctgcctcttctgatcatatcatccctttggattattatcgtacggtgcaacactcgtacaaagatatccatatcccctttgtaagtcgacatgctaccgttgcttctgtgctgcacctagatggagacggttggtgtggcttgggccctgagatcccgctcttctgcgcttctctggcaatggagatagggaaatatgtggcaattccggttttggcagatgcatttctggtcttgtttccggacctgcttcagaatgtcgtttgttttcgacccagaaattacgaaccgccggatttgttctcggttcttctggcccgattagcgacaagtctgacgattcttaactcggcccatctattgagctcatgggtcatcctgtgtccttctcactagccaaaccagccgttttgcgtcgcgccgaatgctctgcccaccaaattaccacgccccatccctatcgcctttcacaccacgtcacttaacgtcagatcccacgttagccccccatgccttttctatacctcacgaaagagtctccgttggcctgcataacaagtgtccgcagaacagcctgatatccatccctgttatcagtagtgggatctacaatcgttcgatcaattgagatatatatgtgtgctggttgtccaatctgcaccacacctgttgtctcatcatcttcattcctttcgcagccagagctaccccgccaagctcttctctcacaaacaacttgctaaccacattcacaatgatttacatggaggccgaatctcactacgaatcttggagcgccttgcccctctttgatcgagttgcgtctcccgatcctgccaaggactttgtcccagatctaaacgactatgaatcaccaacattcgaaatagatcttctctcagaaacttatgactttgacaacttccccacatactctctaccaacggtggattcaaccaagactttgtactccgaagaaccacttgtttgcttcgactttgacttcgcgaacccggctatcgaaaattatataaccacatcgtcgggactgttggacgcagtgccaagccagcttatcgcccttcccaccttcacacggccaagcaaatgcccattccctagttgcaagtcggccacagtctttgaaagcggacgggactttaggcggcattaccggcaacacttcaagcgctttttctgtcgctactcagaatgccctcagtcagctcaagacctgcaagaagtcggcaccaaaggctttgcgactcgcaaggaccgtgctcggcatgagtctaagcacaaaccaacagtgcggtgcccttggcaagacaaggaaggacaacaatgtctgagggtctttagcagggtggataacatgcgagatcactataggcggatacataagtgttgaccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggagggagctggttaacactagattggccaatagcggccgcattgtggctagatgcattcgcgaggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaggcctctgcagtcgacgggcccgggatccgat

SEQ ID NO: 5 - DNA, 7182 bp, Artificial sequence
  Description of artificial sequence Fusarium sporotrichioides 9-2851 Cochliobolus heterostrophus 45-683 Escherichia coli 684-1708 Gibberella zeae 1709-2068 Cochliobolus heterostrophus 2081-2725 Gibberella zeae 2726-4084 Gibberella zeae 4084-4436 (Gibberella zeae 2726-4436) Cloning vector pUC57 4437-7182
cgacagagtcctaggagttgcgttaattaagccgcatttggccagccgctccaccggctgcacatgtcaaggcccacctgcgcattgccatatgaaaatggcctgacatcatgttgaccggcccagcaaacctctgcagagcggtcaagctggcggcgagctttgtagctcgaccacgatgggaatgggaatgcagcagcccagagccatgtgtgccctggtcccccggctcaagtaagccgtggttgtggggatgaaccatccaccgcagcaagttttcttccccattgccctcgtcggccttctgtgagcttctaacacatctgccgcctctgttgactccctctcctcctcccagaccctctggtttgccctcctgtgacctgcacaatctgccccgcgccagtgtcatagtccttggtacgtccccaccaccgcgcgcctccctctctctgtccgctatctcgaccatatcaccaccacagagatagcaccagagtgacgtctcgtgccctgcagcgtgcctcgtgtcgtctgtcttgtcaaccgtcttctcctgcctgccaataccccataccacctcaaatgacccagtcgcaacccttctgcccttaccagccctcggagcttatcgatatctaaccctttgatatagtgcaacaggtgctcagcctacaatcgccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggagggagctggttaacactagtacgcgtgtcgacgccgctccaccggctgcacatgtcaaggcccacctgcgcattgccatatgaaaatggcctgacatcatgttgaccggcccagcaaacctctgcagagcggtcaagctggcggcgagctttgtagctcgaccacgatgggaatgggaatgcagcagcccagagccatgtgtgccctggtcccccggctcaagtaagccgtggttgtggggatgaaccatccaccgcagcaagttttcttccccattgccctcgtcggccttctgtgagcttctaacacatctgccgcctctgttgactccctctcctcctcccagaccctctggtttgccctcctgtgacctgcacaatctgccccgcgccagtgtcatagtccttggtacgtccccaccaccgcgcgcctccctctctctgtccgctatctcgaccatatcaccaccacagagatagcaccagagtgacgtctcgtgccctgcagcgtgcctcgtgtcgtctgtcttgtcaaccgtcttctcctgcctgccaataccccataccacctcaaatgacccagtcgcaacccttctgcccttaccagccctcggagcttatcgatatctaaccctttgatatagtgcaacaggtgctcagcctacaatcgccatggatttcccaaagcctagacaagtccgagagacgagcctgttgatgtactacctagacgtcgtgtttcctctgcaatgcatcaacccaaacaacaattgtctgggaaagagagagtggctgttgactatactgacctctgcgcggcctacgtactatgccacattgtgcatgtcgctcctctataaagaatcgctttcaagcccttgcagatctgaacaggcgatggtatggaagagagagaagacatactactacattcttgcactccaggagtctcagaagctgctgggtgggctcgacaagacatttggcatcacaaggctgaaaggtaccgtcgttgcccttgcttgcatgctacagcttatcagttttgaggtaagacgaatccaccattgtttcgatgctcgatgtcgatgctcgatatccgatctacgattatcgttggtcactaacaaattaaaatagtcttcgcacctaagcaggggagattggcgcgttcacctccatgcggccaacatactcattcctgtcttggttgagggatggtccacagctttgcaatcaggtcccccagccacctccatatggtgcgagctggatgaatcacacttcggctcgactgaagatcaaacctctttgagcttcgaatacgtcggagctttgagattcctgtcaaactcactcgccgcagtcggcatcctgtcttgcatatctattggcccatcagcaccatttgaagattacggccatctcctggaccagccaggtcttatacagctggacgaggtgctggggtgcaggaattggaccatgttgactattctcgaagtgggtaagctggatcgttggaagcgacaggagcaagaacataatcgcttgagcctaaagacgctcgctaggcgcgccatgatgattgaggatatgttgtcagacgagctacaaaggctaccgacagacgagacgcttccagacctcatcactcagatttacgccgcctctatcatgacgtatctgcatacagtagtttccggactcaatcccaacctttcagaggttcaggatagtgtggccgggacgcttcaattgttggagaggctcccaaatcttgaagctgtcacgagcgttacttggcctctagctgtcacaggatgcatggcctcagaaagtcataaggactttttcagaaatactctgaggtcgtatgaggcgacattcagctccttaaaaaagtatgacggaactcttcaggtcttggaagacgcttggaagagaagagagatagatacagagtctccaatgagatgggaagacttgacggatcaccatgggcttccagtgctactttggtagggatggccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggaggtctagattggccaatagcggccgcattgtggctagatgcattcgcgaggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaggcctctgcagtcgacgggcccgggatccgat

SEQ ID NO: 6 - DNA, 1725 bp, Artificial sequence
  Description of artificial sequence Cochliobolus heterostrophus 26-670 Gibberella zeae 671-1327 Gibberella zeae 1328-1681 (Gibberella zeae 671-1681)
gggagctggttaacactagtacgcgtgtcgacgccgctccaccggctgcacatgtcaaggcccacctgcgcattgccatatgaaaatggcctgacatcatgttgaccggcccagcaaacctctgcagagcggtcaagctggcggcgagctttgtagctcgaccacgatgggaatgggaatgcagcagcccagagccatgtgtgccctggtcccccggctcaagtaagccgtggttgtggggatgaaccatccaccgcagcaagttttcttccccattgccctcgtcggccttctgtgagcttctaacacatctgccgcctctgttgactccctctcctcctcccagaccctctggtttgccctcctgtgacctgcacaatctgccccgcgccagtgtcatagtccttggtacgtccccaccaccgcgcgcctccctctctctgtccgctatctcgaccatatcaccaccacagagatagcaccagagtgacgtctcgtgccctgcagcgtgcctcgtgtcgtctgtcttgtcaaccgtcttctcctgcctgccaataccccataccacctcaaatgacccagtcgcaacccttctgcccttaccagccctcggagcttatcgatatctaaccctttgatatagtgcaacaggtgctcagcctacaatcgccatggtttacatggaggccgaatctcactacgaatcttggagcgccttgcccctctttgatcgagttgcgtctcccgatcctgccaaggactttgtcccagatctaaacgactatgaatcaccaacattcgaaatagatcttctctcagaaacttatgactttgacaacttccccacatactctctaccaacggtggattcaaccaagactttgtactccgaagaaccacttgtttgcttcgactttgacttcgcgaacccggctatcgaaaattatataaccacatcgtcgggactgttggacgcagtgccaagccagcttatcgcccttcccaccttcacacggccaagcaaatgcccattccctagttgcaagtcggccacagtctttgaaagcggacgggactttaggcggcattaccggcaacacttcaagcgctttttctgtcgctactcagaatgccctcagtcagctcaagacctgcaagaagtcggcaccaaaggctttgcgactcgcaaggaccgtgctcggcatgagtctaagcacaaaccaacagtgcggtgcccttggcaagacaaggaaggacaacaatgtctgagggtctttagcagggtggataacatgcgagatcactataggcggatacataagtgttgaccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggaggtctagattggccaatagcggccgcattgtggctagcagtttcat

SEQ ID NO: 7 - DNA, 33 bp, Artificial sequence (Gibberella zeae)
  Description of artificial sequence Synthetic primer (Gibberalla zeae)
cacaccatggtttacatggaggccgaatctcac

SEQ ID NO: 8 - DNA, 22 bp, Artificial sequence (Gibberella zeae)
  Descriptioin of artificial sequence Synthetic primer(Gibberalla zeae)
gcatcgcactacagttcgtagc

SEQ ID NO: 9 - DNA, 6509 bp, Artificial sequence
  Description of artificial sequence Cochliobolus heterostrophus 45-683 Escherichia coli 684-1708 Gibberella zeae 1709-2068 Cochliobolus heterostrophus 2067-2705 Gibberella zeae 2706-3363 Gibberella zeae 3364-3722 (Gibberella zeae 2706-3722) Cloning vector pUC57 3799-6509
cgacagagtcctaggagttgcgttaattaagccgcatttggccagccgctccaccggctgcacatgtcaaggcccacctgcgcattgccatatgaaaatggcctgacatcatgttgaccggcccagcaaacctctgcagagcggtcaagctggcggcgagctttgtagctcgaccacgatgggaatgggaatgcagcagcccagagccatgtgtgccctggtcccccggctcaagtaagccgtggttgtggggatgaaccatccaccgcagcaagttttcttccccattgccctcgtcggccttctgtgagcttctaacacatctgccgcctctgttgactccctctcctcctcccagaccctctggtttgccctcctgtgacctgcacaatctgccccgcgccagtgtcatagtccttggtacgtccccaccaccgcgcgcctccctctctctgtccgctatctcgaccatatcaccaccacagagatagcaccagagtgacgtctcgtgccctgcagcgtgcctcgtgtcgtctgtcttgtcaaccgtcttctcctgcctgccaataccccataccacctcaaatgacccagtcgcaacccttctgcccttaccagccctcggagcttatcgatatctaaccctttgatatagtgcaacaggtgctcagcctacaatcgccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggagggagctggttaacctcgagaccggtatctagtacgcgtgtcgacgccgctccaccggctgcacatgtcaaggcccacctgcgcattgccatatgaaaatggcctgacatcatgttgaccggcccagcaaacctctgcagagcggtcaagctggcggcgagctttgtagctcgaccacgatgggaatgggaatgcagcagcccagagccatgtgtgccctggtcccccggctcaagtaagccgtggttgtggggatgaaccatccaccgcagcaagttttcttccccattgccctcgtcggccttctgtgagcttctaacacatctgccgcctctgttgactccctctcctcctcccagaccctctggtttgccctcctgtgacctgcacaatctgccccgcgccagtgtcatagtccttggtacgtccccaccaccgcgcgcctccctctctctgtccgctatctcgaccatatcaccaccacagagatagcaccagagtgacgtctcgtgccctgcagcgtgcctcgtgtcgtctgtcttgtcaaccgtcttctcctgcctgccaataccccataccacctcaaatgacccagtcgcaacccttctgcccttaccagccctcggagcttatcgatatctaaccctttgatatagtgcaacaggtgctcagcctacaatcgccatggtttacatggaggccgaatctcactacgaatcttggagcgccttgcccctctttgatcgagttgcgtctcccgatcctgccaaggactttgtcccagatctaaacgactatgaatcaccaacattcgaaatagatcttctctcagaaacttatgactttgacaacttccccacatactctctaccaacggtggattcaaccaagactttgtactccgaagaaccacttgtttgcttcgactttgacttcgcgaacccggctatcgaaaattatataaccacatcgtcgggactgttggacgcagtgccaagccagcttatcgcccttcccaccttcacacggccaagcaaatgcccattccctagttgcaagtcggccacagtctttgaaagcggacgggactttaggcggcattaccggcaacacttcaagcgctttttctgtcgctactcagaatgccctcagtcagctcaagacctgcaagaagtcggcaccaaaggctttgcgactcgcaaggaccgtgctcggcatgagtctaagcacaaaccaacagtgcggtgcccttggcaagacaaggaaggacaacaatgtctgagggtctttagcagggtggataacatgcgagatcactataggcggatacataagtgttgaccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggaggtctagattggccaatagcggccgcattgtggctagcagtttcatgatctagatgcattcgcgaggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaggcctctgcagtcgacgggcccgggatccgat

SEQ ID NO: 10 - DNA, 43 bp, Artificial sequence (Fusarium sporotrichioides)
  Description of artificial sequence Synthetic primer (Fusarium sporotrichioides)
cacagctagctgatcacgcttagcctcaaacctggtagtatcg

SEQ ID NO: 11 - DNA, 44 bp, Artificial sequence (Fusarium sporotrichioides)
  Description of artificial sequence Synthetic primer (Fusarium sporotrichioides)
cacacccgggttaattaaaggtcagcgatttgaagaagacacgg

SEQ ID NO: 12 - DNA, 10013 bp, Artificial sequence
  Description of artificial sequence Fusarium sporotrichioides 9-2851 Cochliobolus heterostrophus 2869-3507 Escherichia coli 3508-4532 Gibberella zeae 4533-4892 Cochliobolus heterostrophus 4926-5569 Gibberella zeae 5570-6927 Gibberella zeae 6928-7280 (Gibberella zeae 5570-7280) Cloning vector pUC57 7316-10013
ttaattaaaggtcagcgatttgaagaagacacggctggaatgcgtaaactcggtgtaaaacaagttccccagcctccgatcgggtagaagattcccgcaccacactacagagcggctactgcggcgcagagccgggatagccctgtcgcactgagaccgtaacaggtataacctaggcagccatgcaaaagcctgcagtcaagtgggatgcttacaggctcgggtttcaaaaaccagttcacactgcctatcctgtagtaattatccttgttcaaccgtacattctagactaataaaaaacaaaaccaagctatttgtaatcatgataaatggccagtattctcctagcgagtacccgcaaaacatacttccaaaccaccttcggtcactccactagctcaattgagctcagaaaaggcttctgaacctccttcacatccttggatcgaacattcgccaattgcgcgactggtggataagcccactccgaaggagaaacggcgccaacgttggcagcctgctcatagaacttgcagaacttctgagcgtcctcagtcttctcttctttgaccttttcgtatatctcgctaaggcggtacctgcgatcgcacaagtgccacgtgacatagccgtgcatgaagcactcaatcgtgtccatcacctgagggtctttgtccgagaagacagctaccatctgtttggacgagtgcagagtgtcttgggtgagtttctccaaggcttcgtggaggctgatctcatcagagacgacatagttcttgacgagactgatctggtcacgctcatcgtcgaattccttgtagaatgacataagatcattgacccagaccatccagttctccatctgggcaatggctgatgtaatctcaaggaatagacttcgctcattgaactgctccttgggccatagagaagccccgacacaatgacccaagccgttcatacgtcgaagaaactgagggtaatcatgagatcctggaaatcctccaaagttgtactgttcgatccagcatccctcgaaaactataattccatcattgtcaacttcgggatgtggaggaataacaaggccggggtacttacagtccagagtgctgcgaataaggttcaatgagcagaagggaccaaagtgccgaaggacattgggaaagtgctcgttgacgagtgcccaccacggatgggcttgttcgcgtccagcttgaagatcgtcgaaatagtttaccatggttgggtacgggtcatccttgctatcgtccaaaacgagagtgtaggtataatgtatagatagatcggccatgcattctttagagacctttgcccaactgtacacgaccatgccaacgattgtttggagggaagcctgtagtcgcttggggtctaccttgagtagctgctgttggcgtggctgagcaaagtgatgagcagccttgttataagcatagtgcaaattctcgatacgctcttctcgagtgtagttgctatctcggtatcgtatatactcgagaaggcgcacggtagtgttaaggaagtattcggtgggaaagttctccatattggccaaggtgtattggtaacaattcttgatgaatcaacaaactagatcgctatcattgtcttataacaaagtgccgttgctaccaactatatatcattagatggcaaccatctcgaaccgtacactattgctagttgacattttcttgttaggcctaaagactgagccaatatcgatgccaacagcctccccaaccggctacctgtgacatagaccgtgacgtaggcttgatccccctcgtctcatgcaaaactacgaagttgagaaagacttcggctcccagaaggaaggattagtccggtttgtctttagctcgaagagcatttggtttagctacgcaagcgattggtcctggatcctggtgtcaatggacgaggagtaggcctgaagctttgagttcggttgttgtccgtaactgagtttgaactagtggaatagccttctgtcgataggggaggaagcctcacttgtccaagcagccacggagttattttgtgccctgtcaaaagcttaataaggcgaaaggccttaagtgtaaccacttttatgatataccctgcaaacccgtcggctgttacacatattcaaaaccgttcgttcccggatgtactgcaaccgcatctttgaataaggttcaccaacttagccttgtttagttagcgggaggctctgaatttagctgtactatccttgaacccttcctttgtgcgggaatctgtactaagatctccatcacctgtgtctaaaccgcgtatctagcattgtgttctatacacgaactagaacacagatgtcctcggctgtgccgtagataaatcttgttttcgtatttgacatagataacttaacttgcgctaaatggtgattattctccgttgtcgacgcagtacggaattccgaatcccatccttagtcgtactcgtcatagtactgggagattgtccatctaggcaggtgttggagattgagaggtttcttccttcttgacacgtgcttactagtcgccgagacaatcaggcctggggtcgtggggcatgcatgtcagcgtttttgcagttacgagccgcggtgtccttgtttatctacatctaattacgacacaaacgataaccttgcatgccatggctgtcaaagagctagtgcaagttgtgaaaacagcaagtatggcgattaacgttacactacatagtagcaaaatcacaacagaggtagcacagttgatgtgcaagcggaggaggccgcgatactaccaggtttgaggctaagcgtgatcagctaggagttgcgttaattaagccgcatttggccagccgctccaccggctgcacatgtcaaggcccacctgcgcattgccatatgaaaatggcctgacatcatgttgaccggcccagcaaacctctgcagagcggtcaagctggcggcgagctttgtagctcgaccacgatgggaatgggaatgcagcagcccagagccatgtgtgccctggtcccccggctcaagtaagccgtggttgtggggatgaaccatccaccgcagcaagttttcttccccattgccctcgtcggccttctgtgagcttctaacacatctgccgcctctgttgactccctctcctcctcccagaccctctggtttgccctcctgtgacctgcacaatctgccccgcgccagtgtcatagtccttggtacgtccccaccaccgcgcgcctccctctctctgtccgctatctcgaccatatcaccaccacagagatagcaccagagtgacgtctcgtgccctgcagcgtgcctcgtgtcgtctgtcttgtcaaccgtcttctcctgcctgccaataccccataccacctcaaatgacccagtcgcaacccttctgcccttaccagccctcggagcttatcgatatctaaccctttgatatagtgcaacaggtgctcagcctacaatcgccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggagggagctggttaacactagtacgcgtgtcgacgccgctccaccggctgcacatgtcaaggcccacctgcgcattgccatatgaaaatggcctgacatcatgttgaccggcccagcaaacctctgcagagcggtcaagctggcggcgagctttgtagctcgaccacgatgggaatgggaatgcagcagcccagagccatgtgtgccctggtcccccggctcaagtaagccgtggttgtggggatgaaccatccaccgcagcaagttttcttccccattgccctcgtcggccttctgtgagcttctaacacatctgccgcctctgttgactccctctcctcctcccagaccctctggtttgccctcctgtgacctgcacaatctgccccgcgccagtgtcatagtccttggtacgtccccaccaccgcgcgcctccctctctctgtccgctatctcgaccatatcaccaccacagagatagcaccagagtgacgtctcgtgccctgcagcgtgcctcgtgtcgtctgtcttgtcaaccgtcttctcctgcctgccaataccccataccacctcaaatgacccagtcgcaacccttctgcccttaccagccctcggagcttatcgatatctaaccctttgatatagtgcaacaggtgctcagcctacaatcgccatggatttcccaaagcctagacaagtccgagagacgagcctgttgatgtactacctagacgtcgtgtttcctctgcaatgcatcaacccaaacaacaattgtctgggaaagagagagtggctgttgactatactgacctctgcgcggcctacgtactatgccacattgtgcatgtcgctcctctataaagaatcgctttcaagcccttgcagatctgaacaggcgatggtatggaagagagagaagacatactactacattcttgcactccaggagtctcagaagctgctgggtgggctcgacaagacatttggcatcacaaggctgaaaggtaccgtcgttgcccttgcttgcatgctacagcttatcagttttgaggtaagacgaatccaccattgtttcgatgctcgatgtcgatgctcgatatccgatctacgattatcgttggtcactaacaaattaaaatagtcttcgcacctaagcaggggagattggcgcgttcacctccatgcggccaacatactcattcctgtcttggttgagggatggtccacagctttgcaatcaggtcccccagccacctccatatggtgcgagctggatgaatcacacttcggctcgactgaagatcaaacctctttgagcttcgaatacgtcggagctttgagattcctgtcaaactcactcgccgcagtcggcatcctgtcttgcatatctattggcccatcagcaccatttgaagattacggccatctcctggaccagccaggtcttatacagctggacgaggtgctggggtgcaggaattggaccatgttgactattctcgaagtgggtaagctggatcgttggaagcgacaggagcaagaacataatcgcttgagcctaaagacgctcgctaggcgcgccatgatgattgaggatatgttgtcagacgagctacaaaggctaccgacagacgagacgcttccagacctcatcactcagatttacgccgcctctatcatgacgtatctgcatacagtagtttccggactcaatcccaacctttcagaggttcaggatagtgtggccgggacgcttcaattgttggagaggctcccaaatcttgaagctgtcacgagcgttacttggcctctagctgtcacaggatgcatggcctcagaaagtcataaggactttttcagaaatactctgaggtcgtatgaggcgacattcagctccttaaaaaagtatgacggaactcttcaggtcttggaagacgcttggaagagaagagagatagatacagagtctccaatgagatgggaagacttgacggatcaccatgggcttccagtgctactttggtagggatggccgaaggcgagtttggaagtatgttttgcgggtacggatactcgtttggagaatggtggtctgttataatgattacaaatagttcggtcgtgttttgttagaatgaacagttgaacaaggataattacttcggaataggcagttgaaactgaatgtctgtacgtaacctgagcctgtaaccatttcccacttgagtgcaggcttttgcgtaaccaagtctgtacacccgtcggtgcgacagggctacccccaaccctgcaactgcatctgcagctgcagctggcagactggtagactggcgctacgaactgtagtgcgatgcgggaatcttgtacccgctcggaggttggaggtctagattggccaatagcggccgcattgtggctagatgcattcgcgaggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaggcctctgcagtcgacgggcccggg